(19) United States
(12) Patent Application Publication
(10) Pub. No.: US 2015/0019138 A1
(43) Pub. Date: Jan. 15, 2015

(54) SYSTEMS AND METHODS FOR DETERMINING INGREDIENT AMOUNTS FOR PREPARATIONS FOR ADMINISTRATION TO PATIENTS

(71) Applicant: Baxter Corporation Englewood, Englewood, CO (US)

(72) Inventors: Dennis Kaucky, Chicago, IL (US); Darlene Marie Kober, Barrington, IL (US); Douglas Leech, Aurora, CO (US); Dennis A. Tribble, Ormond Beach, FL (US); Elizabeth Ann Zybczynski, Round Lake Beach, IL (US)

(73) Assignee: Baxter Corporation Englewood, Englewood, CO (US)

(21) Appl. No.: 14/387,826

(22) PCT Filed: Mar. 15, 2013
(86) PCT No.: PCT/US13/32421

§ 371 Date: Sep. 24, 2014

Related U.S. Application Data
(60) Provisional application No. 61/618,549, filed on Mar. 30, 2012.
(60) Provisional application No. 61/732,146, filed on Nov. 30, 2012.

(51) Int. Cl. G06F 19/00; G01N 33/15

(52) U.S. Cl. CPC G06F 19/32; G01N 33/15
USPC 702/19

(57) ABSTRACT

Computer systems and non-transitory computer readable media for determining an amount of an ingredient for a preparation for a requested therapy (e.g., an order or a prescription) includes an identifier, volume, and amounts of first and second compounds in each of a plurality of premix solutions. A predefined formulary comprising a formulary concentration of each of a plurality of additions is provided. A premix solution is selected. An amount of a first additions to be added to the premix solution may be selected. A number of bags of the premix solution, needed to satisfy a volume associated with the selected premix solution, are optionally reserved. An additive volume is determined at least by the specified first amounts of the first addition and a corresponding formulary concentration.

### RELATED APPLICATIONS

This application is a §371 national stage entry of PCT Application No. PCT/US2013/032421, which claims priority to U.S. Provisional Patent Application No. 61/618,549 filed Mar. 30, 2012, entitled “SYSTEMS AND METHODS FOR FILLING PRESCRIPTIONS,” and U.S. Provisional Patent Application No. 61/732,146 filed Nov. 30, 2012, entitled “SYSTEMS AND METHODS FOR FILLING PRESCRIPTIONS,” each application being incorporated herein by reference in its entirety.

### TECHNICAL FIELD

The disclosed embodiments relate generally to determining ingredient amounts for a preparation for a requested therapy that is to be administered to a patient. For example, the disclosed embodiments may relate to computer systems and/or non-transitory computer readable media for determining at least one ingredient amount (e.g., a volume) for a preparation (e.g., as a precursor to, for example, filling an order or prescription manually or using a compounder system) by using information related to a number of premix solutions (e.g., in enteral or parenteral bags) and a formulary comprising a formulary concentration of each of a plurality of additions, potential ingredients, or compounds.

### BACKGROUND

A care facility may receive requests for a preparation for a therapy that is to be administered to a patient. As such, the patient care facility may have the need to determine an amount for one or more ingredients for the preparation (e.g., as a precursor to compounding an order or prescription) which typically has been determined by a physician singularly or in conjunction with a dietician, pharmacist or other care provider. The pharmacy may be required to compound large numbers of, for example, IV nutritional formulations also referred to as parenteral nutrition or PN, on a daily basis, which may be challenging. For example, due to injury, disease, or trauma, a patient may need to receive all or some of their nutritional requirements intravenously. In this situation, the patient may receive a parenteral nutritional formulation which, for example, may contain one or more of the following types of chemical ingredients: amino acids, dextrose or carbohydrate, lipid emulsions, vitamins, minerals, electrolytes, etc. Accordingly, there may be a large number of different ingredients or compounds in a typical large-volume parenteral, such as a Total Parenteral Nutrition (TPN) parenteral formulation in which perhaps 30 or more individual ingredients or components may be used.

In this regard, ready to use, pre-packaged, premix formulations (e.g., sometimes in single, double, triple, etc., chamber containers) have been proposed to provide a convenient alternative to compounding all of the potentially many ingredients or compounds in a given preparation. For example, a well-known two chamber bag product containing a solution comprising dextrose or carbohydrate in one chamber and amino acids in a separate second chamber is sold under the trademark CLINIMIX® and CLINIMIX® E (Baxter Healthcare Corporation, Deerfield, Ill.). CLINIMIX products have advanced the field of large-volume parenteral treatment by providing a wide range of physically stable, “ready-to-use”, IV nutrition admixtures manufactured under sterile, high quality conditions. CLINIMIX solutions are indicated as a caloric component in a parenteral nutrition regimen and as the protein (nitrogen) source for offsetting nitrogen loss or for treatment of negative nitrogen balance in patients where: (1) the alimentary tract cannot or should not be used, (2) gastrointestinal absorption of nutrients is impaired, or (3) metabolic requirements for protein are substantially increased, as with extensive burns. FIGS. 1A and 1B respectfully illustrate the composition of various CLINIMIX and CLINIMIX E solutions.

Another well known commercially available ready to use parenteral formulation is OLIMEL™ (Amino Acids, Dextrose and Lipids, with/without Electrolytes) emulsion for infusion, sold by Baxter Healthcare Corporation. OLIMEL is marketed in a triple-chamber bag for parenteral nutrition (PN) and provides adult patients with the essential ingredients of balanced nutrition: protein, carbohydrates and lipids (fats), in a single container, simplifying the preparation of PN for hospitalized patients. The range of OLIMEL formulations, with various concentrations of protein and carbohydrates and an olive oil and soy based lipid emulsion, address the needs of specific patient groups, such as the critically ill, surgery patients and the chronically ill.

OLIMEL is available in multiple formulations, offering one of the highest protein concentration in a multi-chamber bag and formulations that provide a proportionate amount of glucose. OLIMEL is unique in that it contains a lipid in the form of an olive oil and soy based IV fat emulsion, Baxter's proprietary CLINOLEIC. The European Society of Clinical Nutrition and Metabolism (ESPEN) guidelines stress the need to adjust protein and energy formulas based on patients' requirements, as is offered in the OLIMEL formulations. For example, patients who have undergone trauma (such as surgery) experience a breakdown in muscle mass to support healing, so they need protein to help replace the lost muscle mass. Hospitalized patients also need energy supplied by carbohydrates, but supplying too much can lead to hyperglycemia (excessive sugar in the blood), and can impact clinical outcomes. The OLIMEL family of products enables clinicians to match the nutritional therapy to the patient, through a premixed bag that requires fewer steps from preparation to administration.

NUMETA™ was introduced by Baxter Healthcare Corporation at the 22nd Annual European Society of Pediatric and Neonatal Intensive Care (ESPNIC) Medical and Nursing Annual Congress in Hanover, Germany, as the first and only triple-chamber system with formulations specifically designed to meet the range of intravenous (IV) nutritional requirements of neonatal and pediatric patients (preterm newborns through age 18). This premix therapy addresses an important unmet medical need to support neonatal and pediatric patients' changing daily nutritional needs with ready-to-use IV nutrition.

Many preterm infants rely on IV nutrition, also known as parenteral nutrition (PN), at birth to meet all or part of their daily nutritional requirements. Unlike adults, children have a need for growth, which puts them at a particularly high risk of malnutrition because of higher nutritional demands. Acute malnutrition affects almost 25 percent of children admitted to local hospitals, with consequences that include impaired tissue function, suppressed immune systems, defective muscle function and reduced respiratory and cardiac reserve (or capacity).

This ready-to-use nutrition system provides neonatal and pediatric patients with a balanced formulation of amino acids (protein), glucose (carbohydrates), lipids (fats) and electrolytes in a triple-chamber container. NUMETA is designed according to the European Society for Pediatric Gastroenterology, Hepatology and Nutrition (ESPGHAN)-European Society for Clinical Nutrition and Metabolism (ESPEN) Guidelines for Pediatric Parenteral Nutrition to provide well-balanced nutrition that supports the growth of a majority of neonatal and pediatric patients. The ingredients are provided in separate compartments and can be mixed and administered at the point of care, reducing the possible risk of medication errors and contamination, while simplifying the prescription and delivery of parenteral nutrition. Parenteral nutrition formulations can be complex, involving numerous calculations, multiple ingredients, additive dosages and administration rates. In this regard, bags of premix formulations may be a practical option for hospitals to provide standard premix PN for patients in a convenient and easy-to-activate system. The nutritional components are stored in different sections or chambers of a bag, separated by special seals. A clinician breaks the seal between the chambers and gently mixes the admixture or solutions. With multi-chamber bag technology, fewer steps from preparation to administration may reduce the opportunities for error and the potential for touch contamination of the contents. In this regard, the A.S.P.E.N. guidelines support the use of standard formulations to help facilitate a standard process that reduces variation and promotes uniformity among clinicians and healthcare facilities.

Notwithstanding the benefits of premix PN formulations, and despite the fact that they are available in multiple formulations, clinicians may still desire the ability to customize individual patient prescriptions. For example, maintenance vitamins, additional electrolytes and trace elements may not be included, or they may not be present in the desired amounts. Therefore, there is a need to be able to compound ingredients, additives, and/or compounds into premix PN formulations. Methods for reliably determining amounts of an additive or ingredient additions to preparations that utilize premix parenteral solutions are therefore needed. Furthermore, after additions are made there may be a need to ensure that the labeling of such solutions is accurate and efficacious for that customized patient dosage form. Presently, such additions may be determined using hand calculations, and other methods that are prone to error, particularly in the treatment setting, such as at a hospital, where such calculations may be made by fatigued staff. For instance, simple computational error in such compounding with premix parenteral solutions with additions can lead to preparations with mistakes that in turn may lead to incorrectly prepared and/or mislabeled parenteral solutions and the possibility of risk to the patient.

### SUMMARY

In view of the foregoing, described herein are embodiments of systems and methods, in particular computer implemented or computer aided methods, for determining an amount of an ingredient or additive, or amounts of a plurality of ingredients or additives, for a preparation for a requested therapy to be administered to a patient. For example, the requested therapy may include use of a premix solution to which at least a first addition from a plurality of additions may be added. In this regard, in determining the amount (e.g., volume) of the first addition for the preparation for the requested therapy, the embodiments of systems and methods described herein may be operative to determine, in an at least partially computer automated fashion, an additive volume of the first addition to be added to the premix solution to yield the desired preparation for therapy administration to the patient. Such determination may be made with any actual preparation or compounding of the corresponding preparation occurring separately.

In some instances, a premix solution may be provided in one or more predetermined volumes (e.g., such as prepackaged bags containing a certain volume of premix solution). Thus, if an ordered amount of the therapy to be administered differs from the volume of premix solutions available, a determination (e.g., an automated calculation) may be performed to determine the appropriate amount of addition(s) to be added to an available premix solution to satisfy the requested therapy using the premix solution. For such purpose, various approaches are described herein to determine an amount of an ingredient for a preparation of a requested therapy utilizing a premix solution (e.g., including one or more prepackaged bags of premix solution).

In this regard, specific references may be made herein to systems and/or methods for filling a parenteral or enteral prescription. However, it may be appreciated that the systems and methods described in the context of filling a parenteral or enteral prescription may be generally applicable to any requested therapy. For example, systems and methods described herein may be used in determining an amount for one or more ingredients of a preparation for any therapy to be administered that includes a predetermined volume to which ingredients or additions are to be added.

The present disclosure addresses at least some of the limitations and disadvantages described above by providing, for example, computer systems and non-transitory computer readable mediums as well as computer program products for determining an amount of an ingredient, or amounts of a plurality of ingredients, for inclusion in a preparation (e.g., for filling orders or prescriptions for intravenous fluids using semi-automated and automated methods). In this way, any number of amounts for additions may be determined that may be safely and reliably added to a premix parenteral solution, either nutritional (such as CLINIMIX or CLINIMIX E), or purely pharmaceutical, or a combination thereof, while minimizing the introduction of human error that may arise through manual or other ad hoc methods of determining the amount of an addition to be added to a premix solution.

The following presents a summary in order to provide a basic understanding of some of the aspects of the present disclosure. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later. Accordingly, the term aspect, the term embodiment and the term instance are used as synonyms. In other words, one or more features of one or more aspects and/or one or more features of one or more embodiments and/or one or more features of one or more instances described in this application may be separated from the remaining features of the corresponding aspect and/or embodiment and/or instance and combined with separated features of one or more aspects and/or embodiments and/or instances to create new aspects and/or embodiments and/or instances.

As briefly described above, a requested therapy may be provided in the context of an order or prescription (e.g., for a parenteral or enteral prescription or order). Additionally or alternatively, the therapy may generally correspond to any fluid to be administered to a patient including, for example, any intravenous fluid. In this regard, the intravenous fluid may include nutritional components used in the context of parenteral or enteral feeding, or the intravenous fluid may be provided in a context other than parenteral or enteral feeding such as, for example, any context in which an intravenous fluid is to be prepared for administration to a patient. In this regard, the preparation need not be associated with a prescription, but rather may be associated with any requested therapy (e.g., such as an ordered therapy in a patient care facility).

Method embodiments described herein may include receiving a first selection of a premix solution from a plurality of premix solutions and an ordered amount of premix solutions. Each respective one of the premix solutions may include a predetermined description that at least includes an identifier for the respective premix solution, a volume of the respective premix solution a bag, and an amount of a first component in the respective premix solution. The method further includes receiving a second selection that includes a first amount of a first addition. The first addition may be from a plurality of additions, wherein the plurality of additions are predefined in a formulary comprising at least one formulary concentration for each respective addition in the plurality of additions.

The method may further include determining an additive volume for at least the first addition. In this regard, it may be appreciated that selections for, and determinations with respect to, additional additions corresponding to, for example, second additions, third additions, etc., may be provided for in the method, as discussed in greater detail below. In any regard, the determined additive volume that is to be added to the premix solution may be determined at least partially based on the order amount, the volume of the premix solution, the first amount of the first addition, and the formulary concentration for the first addition. For instance, the determination may include considerations relating to differences in volumes between the ordered amount and the premix solution volumes such that the additive volume may reflect and compensate for such differences.

The method, in particular when implemented as a computer implemented method, improves man machine interaction, since a machine based or aided determination of an additive is carried out. Thereby the volume of the additive can be determined at a high precision and erroneous determination of the additive avoided.

A number of feature refinements and additional features are applicable to the foregoing method. These feature refinements and additional features may be used individually or in any combination. As such, each of the following features that will be discussed may be, but are not required to be, used with any other feature or combination of features of the foregoing method.

For example, in some embodiments the ordered amount (i.e., the amount corresponding to volume of the requested therapy to be administered to the patient) may be less than the volume of the premix solution corresponding to the first selection. In such embodiments, the method may further include calculating a partial infusion factor based on the order amount and the volume of the premix solution. Hence, advantageously, man machine interaction is improved, since a machine based or aided calculation of the partial infusion factor is carried out. Thereby the volume of the additive and/or the volume of the requested therapy can be determined at a high precision and erroneous determination of the additive avoided. In turn, the additive volume may at least partially be based on the partial infusion factor. For example, the partial infusion factor may include the quotient of the volume of the premix solution and the ordered amount.

In other embodiments, the order amount may be larger than any one of the volumes of the plurality of premix solutions that are available. As such, the requested therapy may be prepared using a plurality of bags of premix solution. In this regard, the method may further include calculating at least one aliquot volume for the additive volume corresponding to the respective portion of the additive amount for addition to each of the plurality of bags of the premix solution. In an embodiment, the calculating may include aliquoting the additive volume equally between the plurality of bags of premix solution. Hence, advantageously, man machine interaction is improved, since a machine based or aided calculation of the aliquot volume is carried out. Thereby the volume of the aliquot and/or the volume of the requested therapy can be determined at a high precision and erroneous determination of the additive avoided.

Optionally, the method may also include reserving, in response to the first selection, a number of bags of the premix solution needed to satisfy the first selection from a bag inventory. Hence, advantageously, man machine interaction is improved, since a machine based or aided reservation of bags is carried out. Thereby accidental reservation of too many bags and/or accidental use of the wrong number of bags can be avoided. In some implementations, at least one aliquot volume may be determined by dividing the additive volume by the number of bags. In addition, the method may also include establishing a drawdown amount for each one of the number of bags that have been reserved. Further still, the method may include calculating an administration rate for a corresponding bag within the number of bags of been reserved.

In an embodiment, the plurality premix solutions may comprise at least a first premix solution and a second premix solution that have a common corresponding identifier in a different corresponding volume. The first selection may include a common identifier corresponding with the first premix solution and the second premix solution in plurality of premix solutions. In turn, the method may also include selecting automatically, responsive to the first selection, at least one of the first premix solution or at least one of the second premix solution for use in the determining step. For example, the automatic selection may include comparing the amount of the premix solution of the first selection with the stock volume of at least the first premix solution. In this regard, the stock volume of the first premix solution may be less than the stock volume of the second premix solution. In turn, the automatic selection may include selecting the first premix solution when the amount the premix solution comprising the first selection is less than the stock volume of the first premix solution and selecting the second premix solution when the amount of the premix solution of the second selection is greater than the stock volume of the first premix solution. Hence, advantageously, man machine interaction is improved, since a machine based or aided determination of an optimum amount of first and/or second premix solution is carried out.

In another embodiment, the second selection may include a second amount of a second addition from the plurality of additions. In this regard, the determining may include determining an additive volume for the second addition that is to be added to the premix solution. The additive volume for the second addition may at least partially based on the additive volume for the first addition, the ordered amount, the volume of the premix solution, the second amount, and the formulary concentration for the second amount. In this regard, as described to above, a plurality of additions may be selected for a given requested therapy. As such, the additive volume for the first addition and the additive volume for the second addition may affect the total volume. That is, upon the addition of either the first and/or second addition to the premix solution, the total volume of the resulting combination will be larger than the volume of the premix solution prior to the additions. In this regard, the determining step may include iteratively solving for the additive volume of the first and/or second addition based on the affect of the additive volume for the first addition and the additive volume for the second addition on the ordered amount. In other words, advantageously, due to the machine aided method, a precise preparation of the ordered therapy is possible, which would otherwise, i.e., without the method described in this application, not be feasible. In particular, due to the iteration step(s), advantageously the volume of the first addition and/or the second addition can be precisely determined, in order to provide the ordered amount of therapy.

By way of example, in a first iteration, the additive volume for the first addition may be at least partially based on the total volume of the first addition and the volume of the premix solution. Using this total volume, the additive volume for the second addition may be calculated. It may be appreciated that, since the second addition adds additional volume to the previously determined total volume, the calculated additive volume for the first addition may become diluted. That is, the total volume used in the calculation of the additive volume for the first addition in the first iteration may be less than the amount needed to achieve the amount of the first addition of the ordered therapy based on the additional additive volume associated with the second addition. In this regard, the additive volume for the first addition may be recalculated based on the total volume of the ordered amount that includes the portion contributed by the second addition. In turn, the newly determined additive volume for the first addition may be used in recalculating the additive volume for the second addition. This iterative calculation of the additive volumes for the first and second additions may continue until a steady state is reached, until the change in additive volume between iterations is below a predetermined threshold, or until some other appropriate condition is achieved. Furthermore, it may be appreciated that such iterative calculations may extend to the addition of more than two additions. In other words, advantageously, due to the machine aided method, a precise preparation of the ordered therapy is possible in a reasonable amount of time, which would otherwise, i.e., without the method described in this application, not be possible. In particular, due to the iteration step(s) that allow for a determination of one, two or more additions or additives, a steady state of a volume mix can be determined, which would otherwise not be possible. Hence, on the one hand, the user is relieved from the mental task of preparing a therapy and, on the other hand, the precision with which a therapy is determined according to this application is increased.

In an embodiment, at least one of the first selection or the second selection is received through a data input device. For example, the data input device may be an electronic module executed autonomously or may be a user device. Accordingly, in an embodiment, the first selection and the second selection are in the requested therapy and the requested therapy is electronically received. Alternatively, the first selection and the second selection are in the requested therapy and the requested therapy is entered into the computer system through the data input device. In this regard, the data input device may comprise an interactive user interface such that the method also includes receiving user input via use of at least one order template at an interactive interface to enter the requested therapy. Due to the interactive user interface the man machine interaction may be increased, since the at least one order template may provide for desired combinations and/or exclude undesired combinations of additions (e.g., additions that may be undesirable for simultaneous application to a patient). Hence, the likelihood of desirable addition combinations is increased and the risk of undesirable combinations is reduced. In other words, the user may be relieved from or assisted in the mental task to verify the correctness and/or optimization of the therapy that is input through the user interface. Rather, the user interface, by its intrinsic function, may provide such an operation.

In an embodiment, the method may include outputting a manifest output corresponding to the preparation. For example, the manifest output may include at least one of contents of a corresponding bag, including a final amount of the first compound and a final amount of the first addition in the corresponding bag, instructions for adding at least one aliquot volume for the additive volume among a plurality of bags of the premix solution, or instructions for withdrawing a draw down amount from at least one bag of premix solution. In an embodiment, the final amount of the first compound and the final amount of the first addition may each independently expressed in milli-equivalent per milliliter, g/100 mL, percent w/v, percent v/v, milliliters, milligrams, or number of unit doses. Hence, according to this application, in particular to the computer aided method, a therapy can be obtained in a quick and highly reliable manner.

Additionally, the method may include preparing an overlabel for a bag of premix solution comprising the manifest output. Each respective overlabel may specify an expiration date for the preparation and/or each respective overlabel may specify an administration rate for a corresponding bag. Accordingly, the label can be obtained automatically based e.g., on the iterative process described above, so that exact content of the manifest output can be reliable determined by a user from the label. Thus, man machine interaction is improved, since a user can easily verify whether the manifest corresponds to the overlabel. In other words, the user can be relieved from or assisted in the mental task of keeping track of the preparation process of the manifest and can verify the correctness of the result from the overlabel.

In an embodiment, the formulary concentration for an addition in the plurality of additions may be stored in the formulary in unit dose form, milli-equivalent per milliliter form, g/mL form, percent w/v form, percent v/v form, mEq form, mg form, mcg form, IU form, or mL form. An addition in the plurality of additions may be a medication, a trace element, a vitamin, an electrolyte, an IV lipid emulsion, an IV lipid microemulsion, a specialty amino acid, or a nutriceutical ingredient. Furthermore, in an embodiment, the premix solution comprises an amount of a second component in the respective premix solution, and wherein the first component is an amino acid and the second component is dextrose or carbohydrate.

Additionally, systems are described herein for determining an amount of an ingredient for preparation for a requested therapy to be administered to a patient. As explained in greater detail below, the system for determining an amount of an ingredient for preparation for a requested therapy to be administered to a patient may be a computer based system (e.g., including a memory, processor, computer readable program code, etc.). As such, the computer based system may include a user interface executable by a processor of the computer based system. The user interface may be operable to receive a first selection of a premix solution from a plurality of premix solutions and an ordered amount of the premix solution. Each respective one of the premix solutions may include a predetermined description at least comprising:
(i) an identifier for the respective premix solution,
(ii) a volume of the respective premix solution in a bag, and
(iii) an amount of a first component in the respective premix solution.
   The user interface may also be operable to receive a second selection that includes a first amount of a first addition from a plurality of additions. The plurality of additions may be predefined in a formulary comprising at least one formulary concentration for each respective addition in the plurality of additions.

The computer based system may also include a preparation processing module executed by a processor of the computer based system. In this regard, the preparation processing module may be operable to determine an additive volume for the first addition, to be added to the premix solution, at least partially based on the ordered amount, the volume of the premix solution, the first amount, and the formulary concentration for the first addition.

In this regard, it may be appreciated that the computer based system may be operable to execute functionality generally described above with respect to the method for determining an amount of an ingredient for preparation for a requested therapy to be administered to a patient. In some embodiments, the user interface, preparation processing module, and/or other appropriate modules of a computer based may perform, but are not required to perform, any or all of the foregoing functionality described above with respect to the method.

As noted above, embodiments of systems and methods described herein may be at least partially computer automated. In that regard, embodiments may comprise a computer system comprising non-volatile storage, volatile storage, and computational resources for executing an application that determines an amount for an addition for a preparation for a requested therapy (e.g., for fulfilling an order or filling a prescription). The volatile storage and non-volatile storage may collectively comprise one or more data structures, the one or more data structures potentially collectively comprising a description of each premix solution in a plurality of premix solutions where, for each respective premix solution in the plurality of premix solutions, the description of the respective premix solution comprises: (i) an identifier for the respective premix solution, (ii) a volume of the respective premix solution, (iii) an amount of a first compound (e.g., composition or solution) in the respective premix solution, and (iv) an amount of a second compound in the respective premix solution. The one or more data structures further may collectively comprise a formulary comprising a plurality of additions, the formulary comprising a formulary concentration for each respective addition in the plurality of additions.

The volatile storage comprises instructions for receiving a first selection, where the first selection comprises the identifier of a premix solution in the plurality of solutions. The volatile storage further comprises instructions for receiving a second selection, where the second selection comprises a first amount of a first addition in the plurality of additions and a second amount of a second addition in the plurality of additions. The volatile storage optionally comprises instructions for reserving, responsive to the first selection, a number of bags of the premix solution needed to satisfy the first selection. The volatile storage may optionally comprise instructions for determining a plurality of additive volumes, where the plurality of additive volumes comprises at least a first volume and a second volume. The first volume may be determined by: (i) the first amount of the first addition and (ii) the formulary concentration for the addition in the plurality of additions in the formulary that corresponds to the first addition. The second volume may be determined by: (i) the second amount of the second addition and (ii) the formulary concentration for the addition in the plurality of additions in the formulary that corresponds to the second addition.

In some embodiments, an overall target volume for a preparation (e.g., a prescription or order) may be specified. In some such embodiments, an additive volume for a respective addition may be determined by (i) an amount of the respective addition that has been requested to be included in the preparation, (ii) the formulary concentration for the respective addition, and (iii) the overall target volume for the preparation. Alternatively, in some such embodiments, an additive volume for a respective addition may be further determined by (i) an amount of the respective addition that has been requested to be included in the preparation, (ii) the formulary concentration for the respective addition, (iii) the overall target volume for the preparation, (iv) a volume of the premix solution used in the preparation, and (v) a volume of each other addition in the preparation. In some such instances, a volume for each respective additive in the preparation may be computed iteratively as the respective additions in the preparation are processed.

In some embodiments, an overall target energy content for the preparation may be specified. In some such embodiments, an additive volume for a respective addition may be determined by (i) an amount of the respective addition that has been requested to be included in the preparation, (ii) the formulary concentration for the respective addition in the plurality of additions in the formulary that corresponds to the respective addition, and (iii) the overall target energy for the preparation. Alternatively, in some such embodiments, an additive volume for a respective addition may be determined by (i) an amount of the respective addition that has been requested to be included in the preparation, (ii) the formulary concentration for the respective addition in the plurality of additions in the formulary that corresponds to the first addition, (iii) the overall target energy for the preparation, (iv) an energy content of the premix solution used in the preparation, and (v) an energy content of each other addition in the preparation. In some such instances, a volume for each respective additive in the preparation may be computed iteratively as the respective additions in the preparation are processed.

In some embodiments, an overall target mass for the preparation may be specified. In some such embodiments, an additive volume for a respective addition may be determined by (i) an amount of the respective addition that has been requested to be included in the preparation, (ii) the formulary concentration for the respective addition in the plurality of additions in the formulary that corresponds to the respective addition, and (iii) the overall target mass for the preparation. Alternatively, in some such embodiments, an additive volume for a respective addition may be determined by (i) an amount of the respective addition that has been requested to be included in the preparation, (ii) the formulary concentration for the respective addition in the plurality of additions in the formulary that corresponds to the respective addition, (iii) the overall target mass for the preparation, (iv) a mass of the premix solution used in the preparation, and (v) a mass of each other addition in the preparation. In some such instances, a volume for each respective additive in the preparation may be computed iteratively as the respective additions in the preparation are processed.

In some embodiments, an additive volume for a respective addition may be determined by a drug compatibility limit specified between (i) the respective addition and (ii) a compound in the premix solution or another addition in the plurality of additions of the preparation.

In some embodiments, the volatile storage optionally comprises instructions for receiving a first selection, where the first selection comprises the identifier of a premix solution in the plurality of solutions. The volatile storage optionally comprises instructions for receiving a plurality of second selections {1, . . . , N}, where each respective second selection in the plurality of second selections {1, . . . , N} comprises a respective amount of a corresponding addition in the plurality of additions. The volatile storage may include instructions for reserving, responsive to the first selection, a number of bags of the premix solution needed to satisfy the first selection. The volatile storage may comprise instructions for determining a plurality of additive volumes, where each respective additive volume in the plurality of additive volumes corresponds to a second selection in the plurality of second selections. Each respective volume in the plurality of volumes may be determined by: (i) the amount of the addition specified by the corresponding second selection in the plurality of second selections and (ii) the formulary concentration for the addition in the plurality of additions in the formulary specified by the corresponding second selection. In some embodiments, n is 2, 3, 4, 5, 6, 7, 8, 9, 10, 11, 12, 13, 14, 15 or greater than 15.

The volatile storage may optionally comprise instructions for outputting a manifest for the preparation, where the manifest may include instructions for adding a first aliquot of the first volume of the first addition to each bag of the premix solution that has been reserved by the instructions for reserving, where the first aliquot may be calculated by dividing the first volume by the number of bags. The manifest optionally comprises instructions for adding a second aliquot of the second volume of the second addition to each bag of the premix solution that has been reserved by the instructions for reserving, where the second aliquot is calculated by dividing the second volume by the number of bags. The manifest optionally comprises instructions for withdrawing a specific amount from each bag that has been reserved by the instructions. As used herein, a manifest can be in either paper or non-transitory computer readable form.

In some embodiments, the formulary concentration for an addition in the plurality of additions is stored in the formulary in unit dose form, milli-equivalent per milliliter form, g/100 mL form, mg/mL form, percent w/v form, percent v/v form, mEq form, mg form, mcg form, IU form, mL form, or other mass or energy unit definition. In some embodiments, an addition in the plurality of additions is a medication, a trace element, a vitamin, an electrolyte, an amino acid, a nutriceutical, an IV lipid emulsion, or a an IV lipid microemulsion. In some embodiments, the first compound is an amino acid and the second compound is dextrose or carbohydrate. In some embodiments, the manifest is outputted to a user for filling a prescription or fulfilling an order.

In some embodiments, the manifest may assist in compounding the preparation. For example, in an embodiment, the manifest output may be outputted to a compounder for preparing the prescription or fulfilling the order. In such embodiments, the manifest may be outputted in the form of computer readable instructions for operating one or more compounder pumps of the compounder in order to fill the prescription or fulfill the order. In an embodiment, the manifest output may be provided in human readable form (e.g., printed on a label) to assist in manually compounding the preparation.

In some embodiments, the volatile storage and non-volatile storage may collectively comprise instructions for preparing an overlabel for each respective bag in the number of bags that have been reserved for the preparation, where each respective overlabel details the contents of a corresponding bag, including a final amount of the first compound, a final amount of the second compound, a final amount of the first addition, and a final amount of the second addition in the corresponding bag. In some embodiments, the final amount of the first compound, the final amount of the second compound, the final amount of the first addition, and the final amount of the second addition are each independently expressed in milli-equivalent per milliliter, g/100 mL, mg/mL, percent w/v, percent v/v, milliliters, milligrams, number of unit doses, or other mass or energy unit definition. In some embodiments, each respective overlabel details a total volume in a corresponding bag in the number of bags that have been reserved for the preparation. In some embodiments, each respective overlabel details infusion instructions for a corresponding bag in the number of bags that have been reserved for the preparation. In some embodiments, each respective overlabel specifies an expiration date for the preparation. In some embodiments, each respective overlabel specifies an administration rate for a corresponding bag in the number of bags that have been reserved for the preparation. In some embodiments, an overlabel is any label artifact that includes patient and facility identification, final composition and instructions for administration. An overlabel can be either in paper form or electronic form (e.g., a radio frequency identification).

In some embodiments, the manifest may specify that the instructions for withdrawing a specific amount from each bag that has been reserved by the instructions for reserving are to be completed prior to the instructions for adding the first aliquot and the instructions for adding the second aliquot.

In some embodiments, a volume of the respective premix solution is either 1 liter or 2 liters. In some embodiments, a bag in the number of bags comprises a first chamber and a second chamber that are combined to form a single chamber prior to execution of the instructions of the manifest. In some embodiments, a bag in the number of bags comprises a first chamber, a second chamber, and a third chamber that are each combined to form a single chamber prior to execution of the instructions of the manifest. In some embodiments additional chambers may be used and finally combined into a single chamber or they may be kept separate for concomitant administration from multiple chambers, e.g., for physical compatibility reasons.

In some embodiments, a bag in the number of bags comprises a premix bag. As used in some embodiments herein, a premix bag may be a multiple chamber bag (e.g., two chambers, three chambers, four chambers, five chambers, more than five chambers). In such embodiments, the chambers are arranged in any configuration and they can be comingled or administered concomitantly, or in any sequence. Usually the multi-chamber bags are activated to open the sealed compartments to form a single chamber bag and compounding is performed into the activated bag.

It may be appreciated that any of the foregoing features may be applicable to the method described above. These features may be used individually or in any combination. As such, each of the foregoing features may be, but are not required to be, used with any other feature or feature combination of the method described above.

It may be appreciated that functionality for determining an amount of an ingredient for a preparation for a requested therapy may be facilitated by one or more hardware components, computer software components, or combinations thereof. For example, in an embodiment the functionality of determining an amount of an ingredient for a preparation for a requested therapy may be facilitated by a computer executed program comprising computer readable program code. As such, embodiments described herein may include a non-transitory computer readable storage medium (e.g., a computer memory) that may store (e.g., have data physically written onto a portion of the computer memory) such computer readable program code. In this regard, the computer readable program code may be adapted to be executed (e.g., by a processor of a computer system) to perform functionality associated with determining an amount of an ingredient for a preparation for a requested therapy. The computer readable program code may include data structures and/or programs comprising instructions for execution of a processor to provide the functionality described above.

In this regard, one or more data structures that collectively comprise a description of each premix solution in a plurality of premix solutions may be provided where, for each respective premix solution in the plurality of premix solutions, the description of the respective premix solution comprises: (i) an identifier for the respective premix solution, (ii) a volume of the respective premix solution, (iii) an amount of a first compound in the respective premix solution, and (iv) an amount of a second compound in the respective premix solution, a formulary comprising a plurality of additions, where the formulary comprises a formulary concentration for each respective addition in the plurality of additions.

In addition, the computer readable storage medium may include one or more programs for execution by one or more processors of a computer. For instance, the one or more programs may comprise instructions for receiving a first selection, where the first selection comprises the identifier of a premix solution in the plurality of solutions. The one or more programs may further comprise instructions for receiving a second selection, where the second selection comprises a first amount of a first addition in the plurality of additions and a second amount of a second addition in the plurality of additions. The one or more programs may optionally comprise instructions for reserving, responsive to the first selection, a number of bags of the premix solution needed to satisfy the first selection. The one or more programs may further comprise instructions for determining a plurality of additive volumes, where the plurality of additive volumes comprises a first volume and a second volume. The first volume may be determined by: (i) the first amount of the first addition and (ii) the formulary concentration for the addition in the plurality of additions in the formulary that corresponds to the first addition. The second volume may be determined by: (i) the second amount of the second addition and (ii) the formulary concentration for the addition in the plurality of additions in the formulary that corresponds to the second addition.

The one or more programs optionally comprise instructions for outputting a manifest for the preparation (e.g., a prescription or order), where the manifest comprises instructions for adding a first aliquot of the first volume of the first addition to each bag of the premix solution that has been reserved by the instructions for reserving, where the first aliquot is calculated by dividing the first volume by the number of bags. The manifest optionally comprises instructions for adding a second aliquot of the second volume of the second addition to each bag of the premix solution that has been reserved by the instructions for reserving, where the second aliquot is calculated by dividing the second volume by the number of bags. The manifest optionally comprises instructions for withdrawing a specific amount from each bag that has been reserved by the instructions for reserving.

In some embodiments the formulary concentration for an addition in the plurality of additions is stored in the formula in unit dose form, milli-equivalent per milliliter form, g/100 mL form, percent w/v form, percent v/v form, mEq form, mg form, mcg form, IU form, mL form, or another mass or energy unit form. In some embodiments, an addition in the plurality of additions is a medication, a trace element, a vitamin, an electrolyte, or an IV lipid emulsion. In some embodiments, the first compound is an amino acid and the second compound is dextrose or carbohydrate. In some embodiments, the manifest is outputted to a user for filling the prescription or fulfilling the order. In some embodiments, the manifest is optionally output to a compounder for preparing the prescription or fulfilling the order, where the manifest is output in the form of computer readable instructions for operating one or more compounder pumps of the compounder in order to fill the prescription or fulfill the order. In some embodiments, the volatile storage and non-volatile storage may optionally collectively comprise instructions for preparing an overlabel for each respective bag in the number of bags that have been reserved for the preparation, where each respective overlabel details the contents of a corresponding bag, including a final amount of the first compound, a final amount of the second compound, a final amount of a first addition, and a final amount of the second addition in the corresponding bag.

In some embodiments, the final amount of the first compound, the final amount of the second compound, the final amount of the first addition, and the final amount of the second addition are each independently expressed in milli-equivalent per milliliter, g/100 mL, percent w/v, percent v/v, milliliters, milligrams, number of unit doses, or other mass or energy unit form. In some embodiments, each respective overlabel details a total volume in a corresponding bag in the number of bags that have been reserved for the preparation. In some embodiments, each respective overlabel details infusion instructions for a corresponding bag in the number of bags that have been reserved for the preparation. In some embodiments, each respective overlabel specifies an expiration date for the preparation. In some embodiments, each respective overlabel specifies an administration rate for a corresponding bag in the number of bags that have been reserved for the preparation.

In some embodiments, the manifest specifies that the instructions for withdrawing a specific amount from each bag that has been reserved by the instructions for reserving are to be completed prior to the instructions for adding the first aliquot and the instructions for adding the second aliquot. In some embodiments, the volume of the respective premix solution is either 1 liter or 2 liters. In some embodiments, a bag in the number of bags comprises a first chamber and a second chamber that are combined to form a single chamber prior to execution of the instructions of the manifest. In some embodiments, a bag in the number of bags comprises a first chamber, a second chamber, and a third chamber that are each combined to form a single chamber prior to execution of the instructions of the manifest. In some embodiments, additional chambers may be used and finally combined into a single chamber or they may be kept separate for concomitant administration from multiple chambers, e.g., for physical compatibility reasons.

According to an aspect, a computer program product is provided that can be stored on a computer readable medium and/or can be implemented as computer processable data stream, wherein the computer program product comprises computer processable instructions, which instructions when read in the memory of a computer and executed by the computer cause the computer to carry out the method(s) as described in general above, and in more specific examples below.

These computer systems and non-transitory computer readable mediums as well as the computer readable medium provide new, more efficient ways for determining an amount of an ingredient for a preparation that may be provided for manual compounding of the preparation or to a compounder to fill a prescription or fulfill an order. As used herein, the term “non-transitory computer readable media” comprises all computer-readable media, with the sole exception being a transitory, propagating signal.

### BRIEF DESCRIPTION OF THE DRAWINGS

FIGS. 1A and 1B respectively provide the contents of various CLINIMIX and CLINIMIX E solutions in accordance with the prior art.

FIG. 2 is a block diagram of a computer system for executing an application (e.g., computer readable program code stored in non-transitory computer readable medium that is executable by a processor) that fills a prescription according to certain embodiments.

FIG. 3 is a flow diagram illustrating an example of a process performed by a computer system for filling a prescription in accordance with some embodiments.

FIG. 4A illustrates a data structure that details the elements of an exemplary prescription according to certain embodiments.

FIG. 4B illustrates a data structure that details the contents of an exemplary manifest according to certain embodiments in which each bag used to fill a prescription is either not drawn down or is drawn down by the same amount.

FIG. 4C illustrates a data structure that details the contents of an exemplary manifest according to certain embodiments in which each bag used to fill a prescription is drawn down by an independent amount.

FIG. 5 illustrates an exemplary premix solution list in accordance with an embodiment of the present disclosure.

FIG. 6 illustrates an exemplary formulary in accordance with an embodiment of the present disclosure.

FIG. 7A illustrates an interactive user interface screen employable to display and edit a formulary ingredient listing in an embodiment of a prescription order entry system of the present disclosure.

FIG. 7B illustrates an interactive user interface screen employable to initiate creation of a prescription order template for an order comprising a premix solution in an embodiment of a prescription order entry system of the present disclosure.

FIG. 7C illustrates an interactive user interface screen employable to complete or edit a prescription order template for an order comprising a premix solution in an embodiment of a prescription order entry system of the present disclosure.

FIG. 7D illustrates an interactive user interface screen employable to complete and enter a prescription order utilizing a prescription order template in an embodiment of a prescription.

FIG. 7E illustrates an interactive user screen employable to display order content summaries, including an ion content summary.

FIG. 7F illustrates an interactive user screen employable to display order content summaries, including a nutritional content summary.

FIG. 7G illustrates an interactive user screen employable to display order content summaries, including an overall order formula summary.

FIG. 7H illustrates an interactive user interface screen employable to complete and enter a prescription order utilizing a prescription order template in an order entry system of the present disclosure.

FIG. 7I illustrates a formula listing corresponding with the prescription order of FIG. 7H.

Like reference numerals refer to corresponding parts throughout the several views of the drawings.

### DESCRIPTION OF EMBODIMENTS

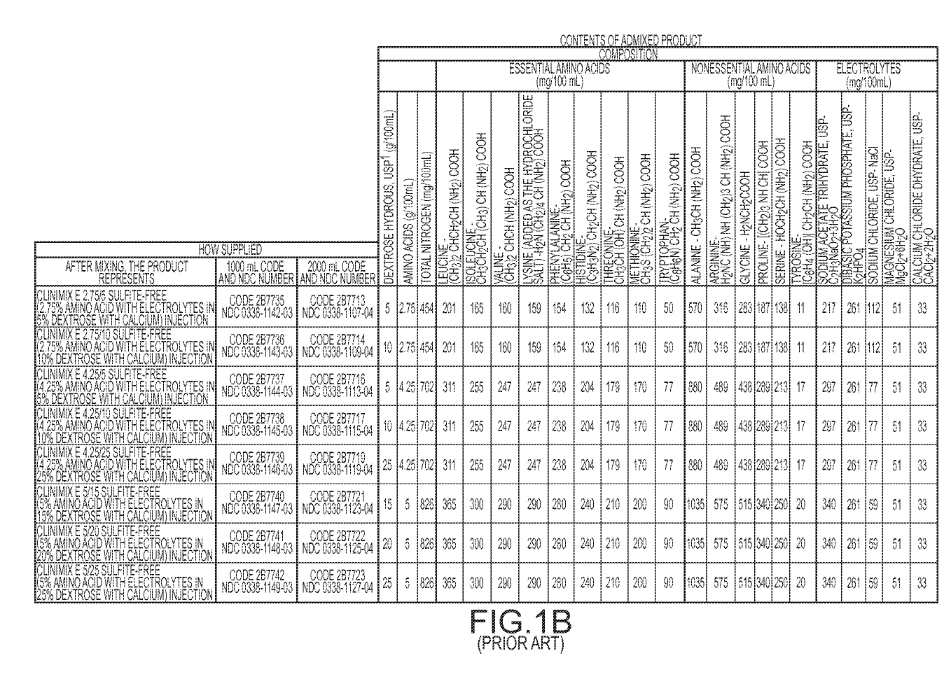

FIG. 2 is a block diagram of a computing network 11 for executing an application that may determine an amount of an ingredient for a preparation (e.g., for use in filling a prescription or fulfilling an order) in accordance with an embodiment of the present disclosure. Thus, while the follow discussion may describe the particular context of determining an amount of an ingredient for a prescription, the disclosure may be generally applicable to determination of an amount for an ingredient of any preparation for any requested therapy, such as a prescription or an order. In any regard, the computing network 11 preferably comprises a computer system 10 having:
one or more central processing units 22;
a main non-volatile (non-transitory) storage unit 14, for example a hard disk drive, for storing software and data, the storage unit 14 controlled by storage controller 12;
a system memory 36, preferably high speed random-access memory, such as DRAM, SRAM, DDR RAM or other random access solid state memory devices, for storing system control programs, data, and application programs, comprising programs and data loaded from non-volatile storage unit 14; system memory 36 may also include read-only memory (ROM);
a user interface 32, comprising one or more input devices (e.g., keyboard 28, a mouse) and a display 26 or other output devices;
optionally, a network interface card 20 (communications circuitry) for connecting to any wired or wireless communication network 34 (e.g., a wide area network such as the Internet);
a power source 24 to power the aforementioned elements; and
one or more communication buses 30 for interconnecting the aforementioned elements of the system.

It will be appreciated that computer system 10 may be part of an apparatus such as a compounder. Alternatively, the computer system 10 may be provided independently for determining an amount of an ingredient for a preparation. Furthermore, in some embodiments, the computer system 10 is a tablet or handheld computer and memory 14 is a memory chipset and there is no controller 12. The one or more communication buses 30 may include circuitry, also referred to as a chipset that interconnects and controls communications between system components. Memory 36 may include high speed random access memory such as DRAM, SRAM, DDR RAM or other random access solid state memory devices. Memory 14 may include mass storage that is remotely located from the one or more central processing units 22. Memory 36, or alternately the non-volatile memory devices within memory 36, comprises a non-transitory computer readable storage medium as described above. In some embodiments, memory 36 or the computer readable storage medium of memory 36 stores the following program of instructions, modules and data structures, or a subset thereof:
operating system 40, which is executed by one or more central processing units 22, comprising instructions for controlling operation of computer 10;
a file system 42 comprising instructions for controlling access to the various files and data structures;
a preparation processing module 44 comprising instructions for determining an amount of an ingredient for a preparation for use in filling a prescription;
a premix solution list 46 comprising a plurality of premix solutions where, for each respective premix solution 48 in the plurality of premix solutions, the premix solution list 46 comprises: (i) an identifier 50 for the respective premix solution 48, (ii) a premix volume 52 of the respective premix solution, (iii) an amount of a first compound 54 in the respective premix solution, and (iv) an amount of a second compound 56 in the respective premix solution;
a formulary 56 comprising a plurality of additions 58, wherein the formulary comprises a formulary concentration 60 for each respective addition 58 in the plurality of additions;
a bag inventory 62 that details a number of bag types and, for each bag type 64, the premix solution identifier 66, the volume 68 of the bag, and the number of available bags 70 in the stock on hand;
one or more prescriptions 72 to be filled; and
one or more manifest 74, each manifest corresponding to a prescription 72 and comprising instructions for filling the prescription using bags from the bag inventory and additions 58 from the formulary 56.

As used herein, the term “premix solution” refers to a solution comprising an amount of a first compound 54 and/or an amount of a second compound 56 that was purchased or otherwise obtained in a state in which the full set of ingredients of the solution are present in the purchased or otherwise obtained product. As such, a “premix solution” is a solution that does not require the addition of components of the solution, because they have already been included in the solution at some predetermined time prior to obtaining or purchasing the premix solution.

In some embodiments, the programs of instructions or modules identified above correspond to sets of instructions for performing a function described above. The sets of instructions can be executed by one or more processors (e.g., processors 22). Each of the above-identified elements may be stored in one or more of the previously mentioned memory devices, and corresponds to a set of instructions for performing a function described above. That is, the memory 36 may include one or more of the above-identified elements in a predefined and/or predetermined manner prior to execution to perform a function described above. The above-identified modules or programs (i.e., sets of instructions) need not be implemented as separate software programs, procedures or modules, and thus various subsets of these modules may be combined or otherwise re-arranged in various embodiments. In some embodiments, memory 36 may store a subset of the modules and data structures identified above. Furthermore, memory 36 may store additional modules and data structures not described above. For instance, patient identifying data such as a patient ID code and patient type (e.g., premature, neonatal, pediatric or adult, etc).

As illustrated in FIG. 2, computer 10 comprises data such as premix solution list 46, formulary 56, and bag inventory 62. Such data can be stored in any form of data storage system including, but not limited to, a flat file, a relational database (SQL), or an on-line analytical processing (OLAP) database (e.g., MDX and/or variants thereof). In some embodiments, premix solution list 46, formulary 56, and bag inventory 62 is stored in a single database. In other embodiments, premix solution list 46, formulary 56, and bag inventory 62 in fact are stored in a plurality of databases that may or may not all be hosted by the same computer 10. In such embodiments, some components of premix solution list 46, formulary 56, and bag inventory 62 are stored on computer systems that are not illustrated by FIG. 2 but that are addressable by wide area network 34.

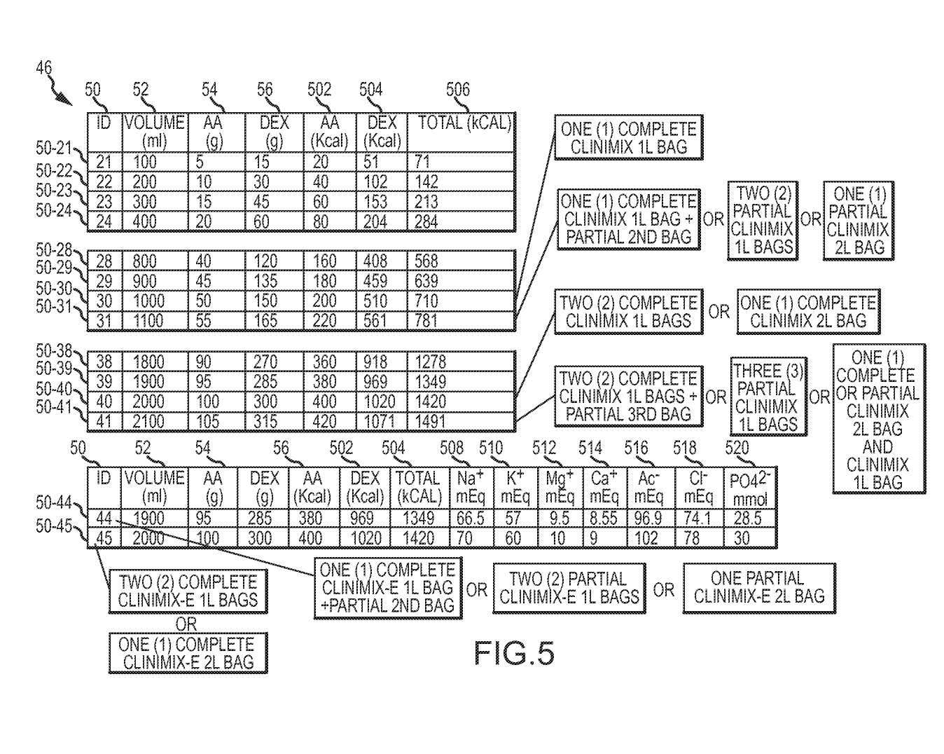

FIG. 5 illustrates a more detailed example of a premix solution list 46 in accordance with an aspect of the disclosure. In the exemplary premix solution list 46, there are 45 different identifiers 50, which identify 45 different premix solution possibilities. In some embodiments, a premix solution 48 includes an associated premix volume 52 such that specification of the premix solution 48, by itself, also specifies a volume of the premix solution as illustrated in FIG. 5. In such embodiments, a user requests a premix solution identifier 50. When this is done, a predetermined amount (e.g., volume) of the identified premix solution is selected. In other embodiments, the premix volume 52 is independent of the premix solution identifier 50. In such embodiments, a user requests a premix solution identifier 50 and also specifies a volume 52. For example, a user may specify a volume, or parameters to determine a volume (e.g., infusion rate and duration parameters), that corresponds with or is otherwise determined utilizing a volume of the corresponding premix solution that the user desires to infuse.

For each premix solution 50 in the exemplary premix solution list 46 of FIG. 5, there is an amount of a first compound 54 in solution that is amino acids in units of grams and an amount of a second compound 56 in solution that is dextrose in units of grams. In the exemplary premix solution list 46 of FIG. 5, the amount of amino acids and the amount of dextrose are also respectively equivalently expressed in units of Kcal (502, 504). In the exemplary premix solution list 46 of FIG. 5, the total Kcal count 506 of each premix solution 48 is also given. As further illustrated in FIG. 5, there is no requirement that each of the premix solutions 48 include the same number of compounds. Indeed, as illustrated in FIG. 5, solutions 44 and 45 include the electrolytes Na⁺ (508), K⁺ (510), Mg⁺ (512), Ca⁺ (514), Ac⁻ (516), Cl⁻ (518), and PO₄²⁻ (520).

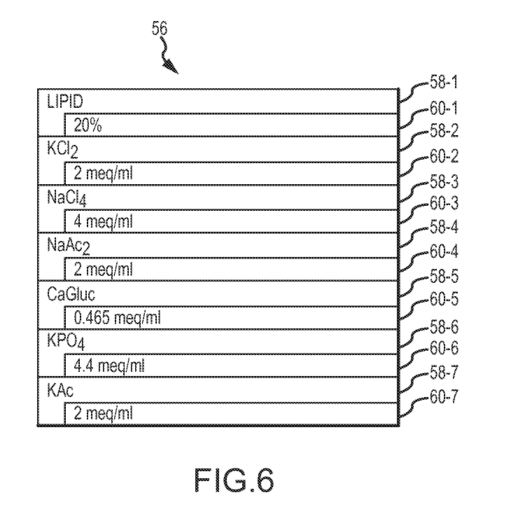

FIG. 6 illustrates a more detailed example of a predetermined formulary 56 in accordance with an aspect of the disclosure. In the exemplary formulary 56, there are 7 different additions 58 (e.g., corresponding to different potential ingredients, compounds, or other additions for addition to a premix solution). For each addition 58, a concentration 60 is specified.

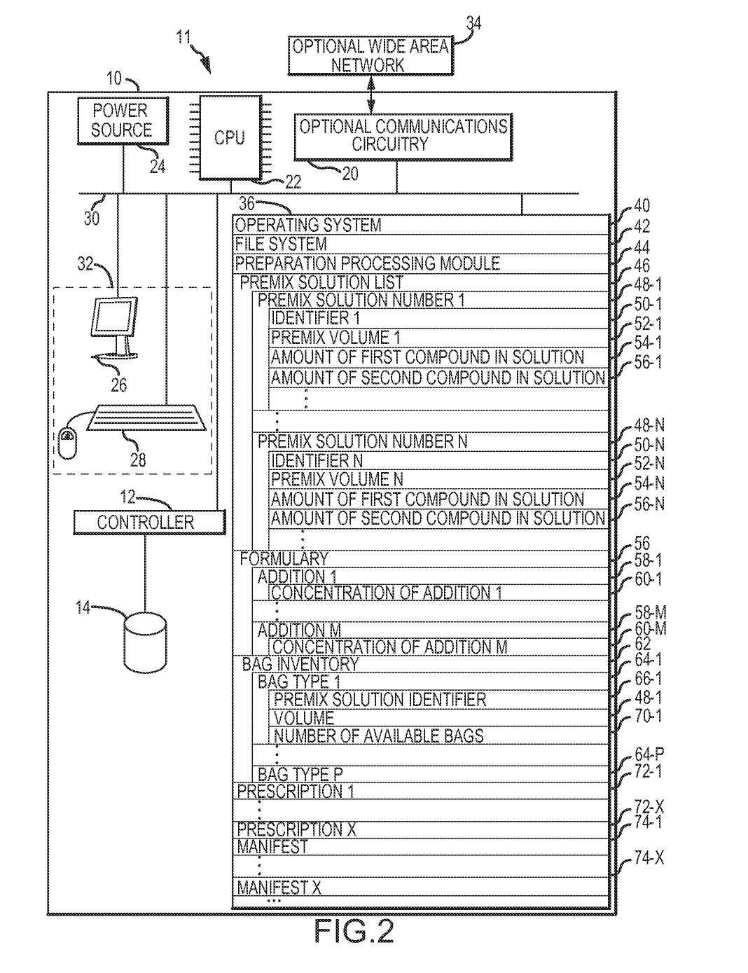

FIG. 3 is a flow diagram illustrating an exemplary process performed by a computer system 10 for determining an amount of an ingredient for a preparation for filling a prescription 72 in accordance with certain embodiments. Referring to FIG. 2, in some embodiments, some or all of the steps set forth below in conjunction with FIG. 3 are performed by prescription processing module 44 using premix solution list 46, formulary 56, and bag inventory 62.

Step 302. In step 302 a first selection is received. The first selection comprises the identifier 50 of a premix solution 48 in a list (plurality) of premix solutions 46 to be used. In some embodiments, the identity of the premix solution 48 is inferred from a prescription 72 that does not explicitly identify the premix solution 48. In such embodiments, step 302 comprises receiving the prescription 72 and determining a suitable premix solution 48 to use in a premix solution list 46. In some embodiments, a premix solution is deemed suitable when it minimizes waste relative to other available premix solutions. In some embodiments, a premix solution is deemed suitable when it minimizes fluid load on a subject relative to other available premix solutions. In some embodiments, a premix solution is deemed suitable when it minimizes costs relative to other available premix solutions. In some embodiments, a premix solution is deemed suitable when it is compatible with other components of a prescription. In some embodiments, a premix solution is deemed suitable based on some function of patient fluid load, waste, cost, and/or compatibility.

In some embodiments, a premix solution 48 includes an associated premix volume 52 such that specification of the premix solution 48, by itself, also specifies a volume of the premix solution. In such embodiments, the first selection includes only the premix solution identifier 50. In other embodiments, the premix volume 52 is independent of the premix solution identifier 50. In such embodiments, the first selection includes both a premix solution identifier 50 and also specifies a volume 52. For example, a user may specify a volume, or parameters to determine a volume (e.g., infusion rate and duration parameters), that corresponds with or is otherwise determined utilizing a volume of the corresponding premix solution that the user desires to infuse.

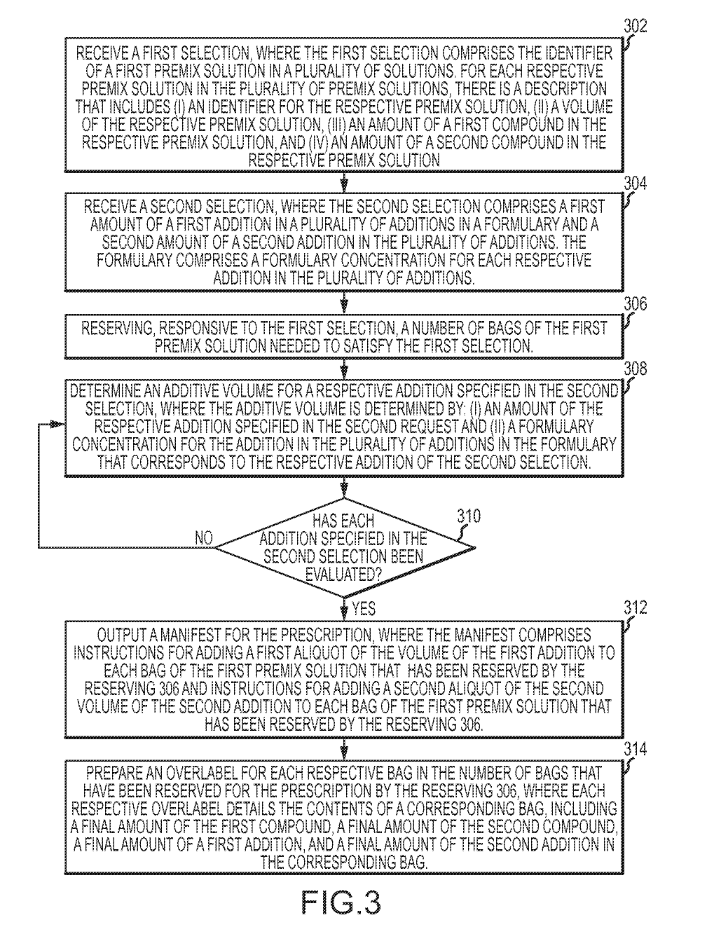

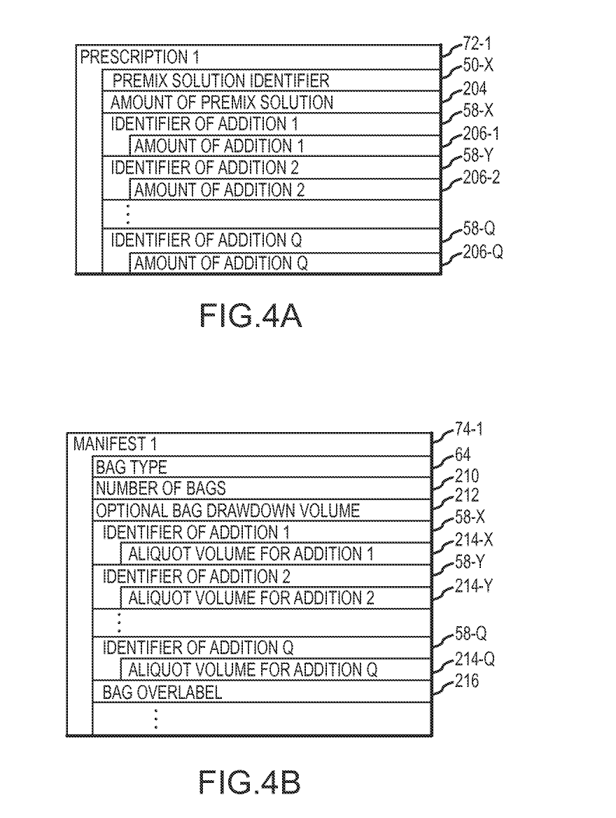

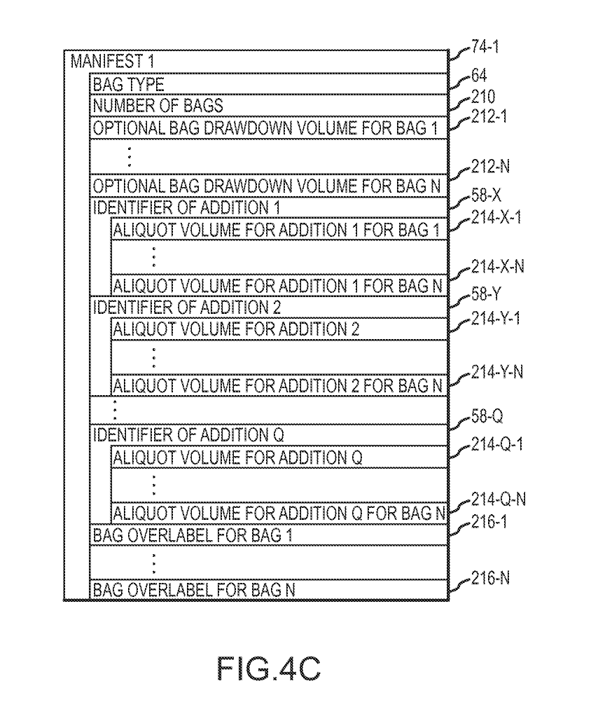

Referring to FIG. 4A, in some embodiments the receiving step 302 is accomplished when a prescription 72 that specifies a premix solution identifier 50 is received. The premix solution identifier 50 specifies which premix solution number 48 in the premix solution list 46 is to be used and the amount 204 of the premix solution number 48 to be used. The prescription 72 may be received by computer 10 electronically (e.g., across optional wide area network 34 or entered manually by a keyboard 28).

For each respective premix solution in the plurality of premix solutions, there is a description that includes: (i) an identifier for the respective premix solution, (ii) a volume of the respective premix solution, (iii) an amount of a first compound in the respective premix solution, and (iv) an amount of a second compound in the respective premix solution. In some embodiments, the first compound is an amino acid and the second compound is dextrose or carbohydrate.

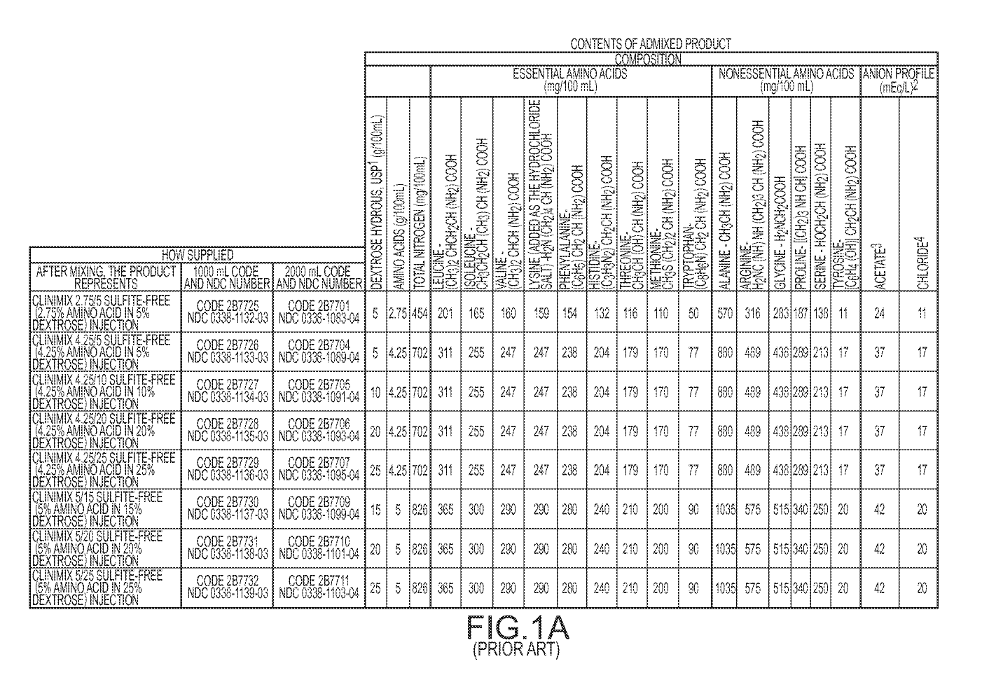

Nonlimiting examples of premix solutions 48 are provided in FIGS. 1A and 1B. In FIGS. 1A and 1B, each row represents a different possible nonlimiting example of a premix solution 48. As illustrated in FIG. 1A, exemplary premix solutions 48 contain between 5% (g/100 mL) and 25% (g/100 mL) dextrose hydrous and between 2.75% (g/100 mL) and 5% (g/100 mL) amino acids. An exemplary premix solution in accordance with the present application comprises between 3% (g/100 mL) and 30% (g/100 mL) dextrose hydrous and one, two, three, four, five, six, seven, eight, nine, ten, eleven, twelve, or thirteen or more of the following amino acids within the specified concentration ranges: between 50 g/100 mL and 250 g/100 mL leucine, between 50 g/100 mL and 200 g/100 mL isoluecine, between 50 g/100 mL and 200 g/100 mL valine, between 50 g/100 mL and 200 g/100 mL lysine, between 50 g/100 mL and 200 g/100 mL phenylalanine, between 50 g/100 mL and 250 g/100 mL histidine, between 50 g/100 mL and 200 g/100 mL threonine, between 50 g/100 mL and 250 g/100 mL methionine, between 25 g/100 mL and 90 g/100 mL tryptophan, between 50 g/100 mL and 800 g/100 mL alanine, between 50 g/100 mL and 450 g/100 mL arginine, between 100 g/100 mL and 350 g/100 mL glycine, between 100 g/100 mL and 300 g/100 mL proline, between 50 g/100 mL and 250 g/100 mL serine, and between 5 g/100 mL and 15 g/100 mL tyrosine.

An example of a specific premix solution 48 is the CLINIMIX 2.75/5 sulfite-free injection of FIG. 1A which contains 5 g/100 mL dextrose hydrous, 201 g/100 mL leucine, 165 g/100 mL isoluecine, 160 g/100 mL valine, 159 g/100 mL lysine, 154 g/100 mL phenylalanine, 132 g/100 mL histidine, 116 g/100 mL threonine, 110 g/100 mL methionine, 50 g/100 mL tryptophan, 570 g/100 mL alanine, 316 g/100 mL arginine, 283 g/100 mL glycine, 187 g/100 mL proline, 138 g/100 mL serine, and 11 g/100 mL tyrosine.

In some embodiments, a volume of the respective premix solution 48 is either one or two liters. In some embodiments, a volume of the respective premix solution is any predetermined volume between 100 mL and 2.5 L (e.g., 100 mL, 200 mL, 300 mL, 400 mL, 500 mL, 600 mL, 700 mL, 800 mL, 900 mL, 1000 mL, 1100 mL, 1200 mL, 1300 mL, 1400 mL, 1500 mL, 1600 mL, 1700 mL, 1800 mL, 1900 mL, 2000 mL, 2100 mL, 2200 mL, 2300 mL, 2400 mL, or 2500 mL).

Step 304.

In step 304, a second selection is received. The second selection comprises a first amount of a first addition 58 in a plurality of additions in a formulary 56 and a second amount of a second addition 58 in the plurality of additions. The formulary 56 comprises a formulary concentration 60 for each respective addition in the plurality of additions. Each addition 58 in the plurality of additions is available in a specific concentration 60 listed in the formulary. In some embodiments, there is more than one available concentration 60 for a given addition 58. In some embodiments, the formulary concentration 60 for an addition 58 in the plurality of additions is stored in the formulary 56 in unit dose form, milli-equivalent per milliliter form, g/mL form, percent w/v form, percent v/v form, mEq form, mg form, mcg form, IU form, or mL form. In some embodiments, an addition 58 in the plurality of additions is a medication, a trace element, a vitamin, an electrolyte, or an IV lipid emulsion.

Referring to FIG. 4A, in some embodiments the receiving step 304 is accomplished when a prescription 72 that specifies a premix solution identifier 50 that specifies which premix solution number 48 in the premix solution list 46 is to be used and the amount 204 of the premix solution number 48 to be used. To satisfy step 304, the prescription further includes an identifier 58-X of a first addition and an identifier 58-Y of a second addition as well as an amount 206-1 of the first addition and an amount 206-2 of the second addition. As illustrated in FIG. 4A, the prescription may include additional identifiers 58 for additional amounts 206 of additions in the formulary 56. The prescription 72 may be received by computer 10 electronically (e.g., across optional wide area network 34 or entered manually by a keyboard 28).

Step 306.

In step 306, a number of bags of the premix solution needed to satisfy the first selection may be reserved. Referring to FIGS. 2 and 4A, this is accomplished by determining the bag type 64 that contains the premix solution 48 identified by the premix solution identifier 50 and comparing the volume 68 of such bags to the amount of premix solution 204 specified in the first selection. For example, if the amount of premix solution 204 specified in the first selection is 1400 mL and the volume 68 of the corresponding bag type 64 is 1000 mL, two bags are reserved. In general, the number of bags that are reserved in step 306 is determined by rounding the quotient obtained by the division of (i) the amount of premix solution 204 specified in the first selection by (ii) the volume 68 of the corresponding bag type 64, and rounding this quotient to the next highest integer.

In optional embodiments, as part of the reserving step 306, a check is made to ensure that a sufficient number of bags of the type needed are available. As illustrated in FIG. 2, bag inventory 62 tracks the number of available bags 70 for each bag type 64. In some embodiments, when the number of available bags is not sufficient, an error condition is called. In some embodiments, this error condition is communicated to a user thereby allowing for the possibility of physically obtaining more bags of the bag type 64 for which there is a shortage in order to fulfill the prescription. In some embodiments, this error condition is communicated to a user and the prescription is not filled until it is resubmitted, presumably at a later date when the bag inventory has been replenished.

In some embodiments, prescription processing module 44, or some equivalent process, periodically polls the number of available bags 70 for each bag type 64 and communicates a report to a user of the number of available bags 70 for each bag type 64. In some embodiments, prescription processing module 44, or some equivalent process, periodically polls the number of available bags 70 for each bag type 64 and communicates a warning when the number of available bags 70 for a given bag type 64 is low. In some embodiments, a determination is made that the number of available bags 70 for a given bag type 64 is low based on a historical usage of such bags. In some embodiments, a determination is made that the number of available bags 70 for a given bag type 64 is low based upon the specific premix solution amounts required to fill a queue of prescriptions 72 that have been submitted to system 11 for filling.

In some embodiments, a bag that is reserved for the prescription comprises a first chamber and a second chamber that are combined to form a single chamber prior to administering to a patient. In some such embodiments, the first chamber holds a dextrose or carbohydrate solution and the second chamber holds one or more amino acids in solution. In some embodiments, the first chamber holds a dextrose or carbohydrate solution and the second chamber holds a solution that comprises leucine. In some embodiments, the first chamber holds an amount of dextrose such that, when the first and second chambers are combined, the resulting premix solution 48 has between 5 and 25 (g/100 mL) dextrose. In some embodiments, the second chamber holds an amount of amino acids such that, when the first and second chamber are combined, the resulting premix solution has between 2.75 (g/100 mL) and 5.00 (g/100 mL) amino acids. In a typical sequence of events, the first and second chambers are combined and then the two or more additions are added to the bag. Then the bag is administered to a patient. Examples of premix solutions suitable for the two chamber bag are the CLINIMIX solutions of FIG. 1A.

In some embodiments, a bag in the number of bags comprises a first chamber, a second chamber and a third chamber that are each combined to form a single chamber prior to administering to a patient. In some such embodiments, the first chamber holds a dextrose or carbohydrate solution, the second chamber holds an amino acid solution, and the third chamber holds a lipid emulsion. In some embodiments, the first chamber holds a dextrose or carbohydrate solution, the second chamber holds a solution comprising leucine, and the chamber holds a solution comprising sodium. In some embodiments, the first chamber contains an amount of dextrose or carbohydrate such that when the three chambers combine the resulting premix solution 48 comprises between 5% (g/100 mL) and 25% (g/100 mL) dextrose, the second chamber contains an amount of amino acids such that when the three chambers combine the resulting premix solution 48 comprises between 2.75% (g/100 mL) and 5.00% (g/100 mL) amino acids, and the third chamber contain an amount of total nitrogen such that when the three chambers combine, the resulting premix solution 48 comprises between 454 and 702 (mg/100 mL) total nitrogen. In some embodiments, the first chamber contains an amount of dextrose or carbohydrate such that when the three chambers combine the resulting premix solution 48 contains between 3 and 30% (g/100 mL) dextrose, the second chamber contains an amount of amino acids such that when the three chambers combine the resulting premix solution 48 comprises between 2.00% (g/100 mL) and 5.50% (g/100 mL) amino acids, and the third chamber contains an amount of total nitrogen such that when the three chambers combine the resulting premix solution 48 comprises between 300 and 800 (mg/100 mL) total nitrogen. Examples of premix solutions suitable for the three chamber intravenous bag are the CLINIMIX E solutions of FIG. 1B. In some embodiments, the third chamber contains amounts of sodium acetate trihydrate, dibasic potassium phosphate, sodium chloride, and magnesium chloride calcium chloride dehydrate such that, when the three chambers combine, the resulting premix solution 48 comprises between 100 (mg/100 mL) and 400 (mg/100 mL) sodium acetate trihydrate, between 100 (mg/100 mL) and 300 (mg/100 mL) dibasic potassium phosphate, between 50 (mg/100 mL) and 150 (mg/100 mL) sodium chloride, between 25 (mg/100 mL) and 100 (mg/100 mL) magnesium chloride, and/or between 25 (mg/100 mL) and 100 (mg/100 mL) calcium chloride dehydrate. In a typical sequence of events, the three chambers are combined and then the two or more additions are added to the intravenous bag. Then the intravenous bag is administered to a patient. Examples of premix solutions suitable for the three chamber intravenous bag are the CLINIMIX E solutions of FIG. 1B.

Step 308.

An additive volume for a respective addition 58 specified in the second selection is determined. In some embodiments, the additive volume may be determined by at least: (i) an amount 206 of the respective addition 58 specified in the second request and (ii) a formulary concentration 60 for the addition 58 in the plurality of additions in the formulary 56 that corresponds to the respective addition 58 of the second selection. For instance, consider the case where the desired amount 206 of the respective addition is 500 mg and the formulary concentration 60 of the addition 58 is 100 mg/mL. In this case, the additive volume is 500 mg×(1 mL/100 mg)=5 mL.

Step 310.

In step 310, a determination may be made as to whether each addition 58 specified in the second selection has been evaluated. If so (310—Yes), process control continues to step 312. If not (310—No), process control returns to step 308. In some embodiments, the second selection comprises between 2 and 5 additions 58. In some embodiments, the second selection comprises between 3 and 10 additions 58. In some embodiments, the second selection comprises more than 5 additions 58.

Step 312.

Step 312 is reached when volumes for a premix solution 48 and two or more additions 58 specified by a prescription 72 have been calculated. Moreover, what is known in step 312 may be the number of bags 70 of the premix solution 48 that will be needed to fill the prescription 72. In step 312, a manifest 74 for the corresponding prescription 72 is optionally outputted. The manifest 74 may comprise instructions for adding a first aliquot of the volume of the first addition 58-X to each bag of the premix solution 50 that has been reserved by the reserving 306. The manifest may comprise instructions for adding a second aliquot of the second volume of the second addition 58-Y to each bag of the premix solution 50 that has been reserved by the reserving 306.

In some embodiments, a draw down volume 212 may be specified by the manifest 74. For example, if the prescription calls for 1200 mL of premix solution whereas the bag inventory 62 indicates that the volume 48 of such bags is 1 L, two bags with a total draw down amount of 800 mL is required. In some embodiments, this draw down volume 212 is equally apportioned between each bag. Thus, in the immediate example, 400 mL is drawn from each of the two bags so that the two bags each contain 800 mL of premix solution, (i.e., ½ of the prescription amount of the premix solution). In some embodiments, the draw down amount is apportioned fully to the last bag. Thus, in the immediate example, 800 mL is drawn from one of the two bags so that one bag contains 1000 mL of premix solution while the other bag contains 200 mL of premix solution.

In some embodiments, the draw down amount is apportioned fully to the last bag provided that the last bag includes a minimum threshold volume of the premix solution. Thus, in the immediate example, consider the case where the minimum threshold volume of the premix solution is 500 mL. In one implementation of the example in accordance with this embodiment, 500 mL is drawn from the second of the two bags so that the second bag has the minimum 500 mL of premix solution and 300 mL is drawn from the first of the two bags so that the first of the two bags contains 700 mL of solution. In a different implementation of the example in accordance with this embodiment, 400 mL is drawn from the first of the two bags so that the first bag has 600 mL of premix solution and 400 mL is drawn from the second of the two bags so that the second of the two bags also contains 600 mL of solution.

In some embodiments, the manifest 74 may specify that the drawn down volume 212 is drawn from each bag that has been reserved for the prescription 72 prior to adding any of the additives 58. In some embodiments, one bag is required to fulfill a prescription. In some embodiments, two bags are required to fulfill a prescription. In some embodiments, three bags are required to fulfill a prescription. In some embodiments, four or more bags are required to fulfill a prescription.

In some embodiments, the manifest is outputted to a user for preparing the prescription 74. In some embodiments, the manifest is output to a compounder for preparing the prescription, where the manifest 74 is output in the form of computer readable instructions for operating one or more compounder pumps of the compounder in order to fill the prescription 72.

FIG. 4B illustrates an exemplary manifest 74 in accordance with an embodiment of the present disclosure in which there is either no draw down amounts or equal drawdown amounts specified for each bag. The manifest 74, which corresponds to a prescription 72 to which the manifest corresponds, details an bag type 64 that is available in the bag inventory 64. The manifest 74 indicates a number 210 of bags of the bag type that have been reserved. The manifest further details an optional bag drawdown volume 212 which is the volume of premix solution 48 within the bags that is to be drawn out of each of the bags prior to adding additions 58.

Continuing to refer to FIG. 4B, for each respective addition 58 to be added to fulfill the corresponding prescription 72, the manifest 74 includes an identifier of the respective addition 58 and a per bag aliquot volume 214 of the respective addition 58 to be added to each bag. The manifest 74 further includes a bag overlabel that is described in further detail below in the description of step 314.

FIG. 4C illustrates an exemplary manifest 74 in accordance with an embodiment of the present disclosure in which each bag is drawn down by an independent amount. As was the case of FIG. 4B, the manifest 74, which corresponds to a prescription 72 to which the manifest corresponds, details an bag type 64 that is available in the bag inventory 64. The manifest 74 indicates a number 210 of bags of the bag type that have been reserved. The manifest 74 further details a bag drawdown volume 212 for each bag. In other words, the manifest specifies an independent drawdown volume 212 for each respective bag which is the volume of premix solution 48 to be drawn from the respective bag prior to adding additions 58. For example, consider the case where the prescription 72 calls for 1200 mL of premix solution 48 whereas the bag inventory 62 indicates that the volume 48 of such bags is 1 L. Thus, two bags with a total draw down amount of 800 mL are required. If the draw down amount is apportioned fully to the second bag, then the drawdown volume 212-1 for bag 1 will be 0 mL and the drawdown volume 212-2 for bag 2 will be 800 mL so that the first bag contains 1000 mL of premix solution while the second bag contains 200 mL of premix solution.

Continuing to refer to FIG. 4B, for each respective addition 58 to be added to fulfill the corresponding prescription 72, the manifest 74 includes an identifier of the respective addition 58 and an independent per bag aliquot volume 214 of the respective addition 58 to be added to each bag. In the example given above, where the first bag contains 1000 mL of premix solution and the second bag contains 200 mL of premix solution, the per bag aliquot volume 214-1 for the first bag will be five times greater than the per bag aliquot volume 214-2 for the second bag. Continuing to refer to FIG. 4C, in instances where each bag to be used to fill a prescription 72 has an independent drawn down amount (volume) 212, the manifest 74 further includes an independent bag overlabel 216 for each bag.

Step 314.

In step 314, an optional overlabel 216 for each respective bag in the number of bags that have been reserved for the prescription 72 by the reserving step 306 is prepared. Each respective overlabel 216 details the contents of a corresponding bag, including a final amount of the first compound in the premix solution, a final amount of the second compound in the premix solution, a final amount of a first addition, and a final amount of the second addition in the corresponding bag.

In some embodiments, the final amount of the first compound, the final amount of the second compound, the final amount of the first addition, and the final amount of the second addition are each independently expressed in milli-equivalent per milliliter, g/mL, percent w/v, percent v/v, milliliters, milligrams, number of unit doses, or other designated units.

In some embodiments each respective overlabel 216 details a total volume in a corresponding bag in the number of bags that have been reserved for the corresponding prescription 72. In some embodiments, each respective overlabel 216 details infusion instructions for a corresponding bag in the number of bags that have been reserved for the prescription. In some embodiments, each respective overlabel 216 specifies an expiration date for the corresponding prescription 72. In some embodiments, each respective overlabel 216 specifies an administration rate for a corresponding bag in the number of bags that have been reserved for the prescription.

The methods illustrated in FIG. 3 may be governed by programs of instructions that are stored in a computer readable storage medium and that are executed by at least one processor of at least one computer as described above. Each of the operations shown in FIG. 3 may correspond to a program of instructions stored in a non-transitory computer memory or computer readable storage medium. In various implementations, the non-transitory computer readable storage medium includes a magnetic or optical disk storage device, solid state storage devices such as Flash memory, or other non-volatile memory device or devices. The computer readable instructions stored on the non-transitory computer readable storage medium may be in source code, assembly language code, object code, or other instruction format that is interpreted and/or executable by one or more processors.

In some embodiments, an overall target volume for the prescription may be specified. In some such embodiments, an additive volume for a respective addition calculated in step 308 may be determined by (iii) the overall target volume for the prescription.

In some embodiments, an overall target volume for the prescription may be specified. In some such embodiments, an additive volume for a respective addition calculated in step 308 may be determined by (iii) the overall target volume for the prescription, (iv) a volume of the premix solution used in the prescription, and (v) a volume of each other addition in the prescription. In some such instances, a volume for each respective additive in the prescription may be computed iteratively as the respective additions in the prescription are processed by instances of step 308.

In some embodiments, an overall target energy content for the prescription may be specified. In some such embodiments, an additive volume for a respective addition calculated in step 308 may be determined by (iii) the overall target energy for the prescription.

In some embodiments, an overall target energy content for the prescription may be specified. In some such embodiments, an additive volume for a respective addition calculated in step 308 may be determined by (iii) the overall target energy for the prescription, (iv) an energy content of the premix solution used in the prescription, and (v) an energy content of each other addition in the prescription. In some such instances, a volume for each respective additive in the prescription may be computed iteratively as the respective additions in the prescription are processed by instances of step 308.

In some embodiments, an overall target mass for the prescription may be specified. In some such embodiments, an additive volume for a respective addition calculated in step 308 may be determined by (iii) the overall target mass for the prescription.

In some embodiments, an overall target mass for the prescription may be specified. In some such embodiments, an additive volume for a respective addition calculated in step 308 may be determined by (iii) the overall target mass for the prescription, (iv) a mass of the premix solution used in the prescription, and (v) a mass of each other addition in the prescription. In some such instances, a volume for each respective additive in the prescription may be computed iteratively as the respective additions in the prescription are processed by instances of step 308.

In some embodiments, an additive volume for a respective addition specified in the second selection may be determined by a drug compatibility limit specified between (i) the first addition and (ii) a compound in the premix solution or another addition in the plurality of additions of the prescription.

In some embodiments, deviation tolerances to the overall target volume, overall target energy content, or overall target mass are permitted.

In some embodiments, the deviation tolerance to the overall target volume is within ±0.05 percent, within ±0.1 percent, within ±1.0 percent, within ±2.0 percent, within ±4.0 percent, within ±6.0 percent, within ±10.0 percent, or within ±20.0 percent of the specified overall target volume.

In some embodiments, the deviation tolerance to the overall target energy content is within ±0.05 percent, within ±0.1 percent, within ±1.0 percent, within ±2.0 percent, within ±4.0 percent, within ±6.0 percent, within ±10.0 percent, or within ±20.0 percent of the specified overall energy content.

In some embodiments, the deviation tolerance to the overall target mass is within ±0.05 percent, within ±0.1 percent, within ±1.0 percent, within ±2.0 percent, within ±4.0 percent, within ±6.0 percent, within ±10.0 percent, or within ±20.0 percent of the specified overall target mass.

Now that an exemplary method in accordance with the present disclosure has been given with respect to FIG. 3, examples of these processing steps will be given using the exemplary premix solution list 46 set forth in FIG. 5 and the exemplary formulary 56 set forth in FIG. 6. In these examples set forth below, the corresponding processing steps of FIG. 3 may be used so that the correspondence between the processing steps of the example and the general processing steps of FIG. 3 is easily understood. However, the use of these processing steps in the examples does not serve to limit the scope of any of the general description provided above.

### First Example

Step 302.

In exemplary step 302, a first selection is received. The first selection comprises the identifier of a premix solution 50 in a plurality of premix solutions (the premix solution list 46). In this example, the premix solution is solution 50-40 of FIG. 5 (CLINIMIX 2000 mL, 100 g AA, 300 g dextrose).

Step 304.

In exemplary step 304, a second selection is received. The second selection comprises a first amount of a first addition 58 in the plurality of additions (formulary 56) and a second amount of a second addition 58 in the plurality of additions. In particular, the second selection includes a request for 50 g of lipid, 10 ml of MVI, 1 ml of trace elements, 20 meq of KCl, 40 meq of NaCl, 80 meq of NaAc, and 4.2 meq CaGluc.

Step 306.

In exemplary step 306 and responsive to the first selection, a number of bags 64 of the premix solution needed to satisfy the first selection are reserved from the bag inventory 62. Based on the first and second selections, two one liter (1 L) bags of CLINIMIX 5/15 are used as the starting formulation. No volume is removed from the starting bag. Determinations for an amount of the additions to be added to the premix solution are then made as would be required to fulfill the prescription, as set forth below.

Step 308.

In exemplary step 308, a plurality of additive volumes are determined based on the additions 58 specified in the second selection and the formulary concentrations of these additions. For instance, the plurality of additive volumes comprises a first volume, where the first volume is determined by: (i) the first amount of the first addition (50 g of lipid) and (ii) the formulary 56 concentration for the first addition in the plurality of additions in the formulary that corresponds to the first addition (20 g/100 ml). The plurality of additive volumes comprises a second volume, where the second volume is determined by: (i) the second amount of the second addition (10 ml of MVI) and (ii) the formulary concentration for the second addition in the plurality of additions in the formulary that corresponds to the second addition (unit dose/10 mL). In the present example, step 308 comprises making the calculations set forth in Table 1 below based on the amounts of additions specified in the second request and the corresponding concentrations of such additions in the exemplary formulary 56 illustrated in FIG. 6:

TABLE 1

| | | | |
|---|---|---|---|
| | | | Amount Per 2 |
| | Total | | Intravenous |
| | Volume | Volume | Bags (Dose |
| Desired | to be | to be | Divided 2 |
| Dose | Added | Added | bags per day |

| | | | Formulary | | Converted | to | to Each | for Q2 or |
|---|---|---|---|---|---|---|---|---|
| Addition | Desired | | Concentration | | to | Premix | Premix | twice daily |
| 58 | Dose | | 60 | | Volume | Bags | Bag | administration) |
| Lipid | 50 | g | 20 | g/100 ml | 250 ml | 250 ml | 125 ml | 25 g |
| MVI | 10 | ml | Unit Dose | | 10 ml | 10 ml | 5 ml | 5 ml |
| Trace | 1 | ml | Unit Dose | | 1 ml | 1 ml | 0.5 ml | 0.5 ml |
| Elements | | | | | | | | |
| KCl | 20 | meq | 2 | meg/ml | 10 ml | 10 ml | 5 ml | 10 meq KCl |
| NaCl | 40 | meq | 4 | meq/ml | 10 ml | 10 ml | 5 ml | 20 meq NaCl |
| NaAcetate | 80 | meq | 2 | meq/ml | 40 ml | 40 ml | 20 ml | 40 meq |
| | | | | | | | | Sodium |
| | | | | | | | | Acetate |
| CaGluc | 4.2 | meq | 0.465 | meq/ml | 9 ml | 9 ml | 4.5 ml | 2.1 meq |
| | | | | | | | | Calcium |
| | | | | | | | | Gluconate |

| Total | N/A | N/A | 330 ml | 330 ml | 165 ml | 165 ml |
|---|---|---|---|---|---|---|
| Additions | | | | | | |

Step 312.

In exemplary step 312, a manifest 74 for the corresponding intravenous prescription 72 is output. This manifest can include, for example, overlabeling, macronutrient ingredients, micronutrient ingredients, patient identification and infusion directions. However, it may also be appreciated that the manifest need not be reduced to hard copy. For example, the manifest may include an output in the form of computer-readable data stored in a memory. Exemplary step 312 is reached when volumes for a premix solution 48 and two or more additions 58 specified by a prescription 72 have been calculated.

Moreover, what is known in step 312 is the number of intravenous bags 70 of the premix solution 48 that will be needed to fill the prescription 72. Specifically, in determining overlabeling for the manifest in this example, it has already been determined that the prescription order will use two intravenous bags of CLINIMIX 5/15 with the additions specified in the second selection being divided between bags. One bag will be infused immediately following the other bag. The total order volume is 2*1000 ml (2 CLINIMIX BAGS)+330 ml (additions 58)=2330 ml. Thus, each bag contains 1000 ml (CLINIMIX)+165 ml (½ of the additions 58) for a total volume of 1165 ml.

In determining macronutrient ingredients for the manifest in this example, the final concentration of amino acids is 50 g AA/1165 ml=4.29%. The final concentration of dextrose is 150 g dextrose/1165 ml=12.88%. The final concentration of lipids is 25 g lipids/1165 ml=2.15%.

In determining the micronutrient ingredients for the manifest in this example, the amount per bag values are determined based on Table 1 above. The per liter values are calculated according to the formula:

$$\begin{matrix}Per Liter Value = \frac{Amount Per Bag Value}{1.165 L} \\ = 0.86 * Amount Per Bag Value\end{matrix}$$

Using this formula and Table 1, the values for Table 2 below are determined.

TABLE 2

| | | | |
|---|---|---|---|
| | Per 2 Bags (for | | |
| Micro- | Q2 or twice daily | | |
| nutrient | administration) | Per Liter | Per Day |

| | | | | | | |
|---|---|---|---|---|---|---|
| MVI | 5 | ml | 4.3 | ml | 10 | ml |
| Trace | 0.5 | ml | 0.43 | ml | 1 | ml |
| K+ | 10 | meq | 8.6 | meq | 20 | meq |
| Na+ | 60 | meq | 51.6 | meq | 120 | meq |
| Ca++ | 2.1 | meq | 1.80 | meq | 4.2 | meq |
| Cl− | 30 | meq | 25.8 | meq | 60 | meq |
| Ac− | 40 | meq | 34.3 | meq | 80 | meq |

The following considerations are made in order to determine the infusion instructions for the manifest in this example. A total volume of 2330 ml is to be infused over 24 hours:

$$\begin{matrix}Infusion Rate = \frac{Infusion Volume}{Infusion Period} \\ = \frac{2330 ml}{24 hrs} \\ = \frac{97 ml}{hr}\end{matrix}$$

Accordingly, each intravenous bag is infused over 12 hours consecutively (97 ml/hr), one after the other (intravenous bag 1 of 2 and intravenous bag 2 of 2/24 hour period).

### Second Example

Step 302.

In exemplary step 302, a first selection is received. The first selection comprises the identifier of a premix solution 50 in a plurality of premix solutions (the premix solution list 46). In this example, the premix solution is solution 50-38 of FIG. 5 (CLINIMIX 1800 mL, 90 g AA, 270 g dextrose).

Step 304.

In exemplary step 304, a second selection is received. The second selection comprises a first amount of a first addition 58 in the plurality of additions (formulary 56) and a second amount of a second addition 58 in the plurality of additions. In particular, the second selection includes a request for 50 g of lipid, 10 ml of MVI, 1 ml of trace elements, 20 meq of KCl, 40 meq of NaCl, 80 meq of NaAc, and 4.2 meq CaGluc.

Step 306.

In exemplary step 306 and responsive to the first selection, a number of intravenous bags 64 of the premix solution needed to satisfy the first selection are reserved from the intravenous bag inventory 62. Based on the first and second selections, two one liter (1 L) bags of CLINIMIX 5/15 are used as the starting formulation. A total of 200 ml is to be removed, 100 ml from each of the two intravenous bags. Determinations for an amount of the additions are then made as would be required to fulfill the prescription, as set forth below.

Step 308.

In exemplary step 308, a plurality of additive volumes are determined based on the additions 58 specified in the second selection and the formulary concentrations of these additions. For instance, the plurality of additive volumes comprises a first volume, where the first volume is determined by: (i) the first amount of the first addition (50 g of lipid) and (ii) the formulary 56 concentration for the first addition in the plurality of additions in the formulary that corresponds to the first addition (20 g/100 ml). The plurality of additive volumes comprises a second volume, where the second volume is determined by: (i) the second amount of the second addition (10 ml of MVI) and (ii) the formulary concentration for the second addition in the plurality of additions in the formulary that corresponds to the second addition (unit dose/10 mL). In the present example, step 308 comprises making the calculations set forth in Table 3 below based on the amounts of additions specified in the second request and the corresponding concentrations of such additions in the exemplary formulary 56 illustrated in FIG. 6:

TABLE 3

| | | | | | | | | Amount Per |
|---|---|---|---|---|---|---|---|---|
| | | | | | | | | Intravenous |
| | | | | | | | | Bag (Dose |
| | | | | | | Total | | Divided |
| | | | | | Desired | Volume to | Volume to | Equally 2 |
| | | | | | Dose | be Added | be Added | bags per day |
| | | | Formulary | | Converted | to | to Each | for Q2 or |
| Addition | Desired | | Concentration | | to | Intravenous | Intravenous | twice daily |
| 58 | Dose | | 60 | | Volume | Bags | Bag | administration) |
| Lipid | 50 | g | 20 | g/100 ml | 250 ml | 250 ml | 125 ml | 25 g |
| MVI | 10 | ml | Unit Dose | | 10 ml | 10 ml | 5 ml | 5 ml |
| Trace | 1 | ml | Unit Dose | | 1 ml | 1 ml | 0.5 ml | 0.5 ml |
| Elements | | | | | | | | |
| KCl | 20 | meq | 2 | meg/ml | 10 ml | 10 ml | 5 ml | 10 meq KCl |
| NaCl | 40 | meq | 4 | meq/ml | 10 ml | 10 ml | 5 ml | 20 meq NaCl |
| NaAcetate | 80 | meq | 2 | meq/ml | 40 ml | 40 ml | 20 ml | 40 meq |
| | | | | | | | | Sodium |
| | | | | | | | | Acetate |
| CaGluc | 4.2 | meq | 0.465 | meq/ml | 9 ml | 9 ml | 4.5 ml | 2.1 meq |
| | | | | | | | | Calcium |
| | | | | | | | | Gluconate |
| Total | N/A | | N/A | | 330 ml | 330 ml | 165 ml | 165 ml |
| Additions | | | | | | | | |

Step 312.

In exemplary step 312, a manifest 74 for the corresponding intravenous prescription 72 is outputted. This manifest can include, for example, overlabeling, macronutrient ingredients, micronutrient ingredients, and infusion directions. Exemplary step 312 is reached when volumes for a premix solution 48 and two or more additions 58 specified by a prescription 72 have been calculated.

Moreover, what is known in step 312 is the number of intravenous bags 70 of the premix solution 48 that will be needed to fill the prescription 72. Specifically, in determining overlabeling for the manifest in this example, it has already been determined that the prescription order will use two intravenous bags of CLINIMIX 5/15 with the additions specified in the second selection being divided between bags. One bag will be infused immediately following the other bag. The total order volume is 2*1000 ml (2 CLINIMIX BAGS)−200 ml (volume removed after intravenous bag activation)+330 ml (additions 58)=2130 ml. Thus, each bag contains 1000 ml (CLINIMIX)−100 ml (volume removed after activation)+165 ml (½ of the additions 58) for a total volume of 1065 ml.

In determining macronutrient ingredients for the manifest in this example, the final concentration of amino acids is 45 g AA/1065 ml=4.23%. The final concentration of dextrose is 135 g dextrose/1065 ml=12.68%. The final concentration of lipids is 25 g lipids/1065 ml=2.35%.

In determining the micronutrient ingredients for the manifest in this example, the amount per bag values are determined based on Table 3 above. The per liter values are calculated according to the formula:

$$\begin{matrix}Per Liter Value = \frac{Amount Per Bag Value}{1.065 L} \\ = 0.94 * Amount Per Bag Value\end{matrix}$$

Using this formula and Table 3, the values for Table 4 below are determined.

TABLE 4

| | | | |
|---|---|---|---|
| | Per 2 Bags (for | | |
| Micro- | Q2 or twice daily | | |
| nutrient | administration) | Per Liter | Per Day |

| | | | | | | |
|---|---|---|---|---|---|---|
| MVI | 5 | ml | 4.7 | ml | 10 | ml |
| Trace | 0.5 | ml | 0.47 | ml | 1 | ml |
| K+ | 10 | meq | 9.4 | meq | 20 | meq |
| Na+ | 60 | meq | 56.3 | meq | 120 | meq |
| Ca++ | 2.1 | meq | 1.97 | meq | 4.2 | meq |
| Cl− | 30 | meq | 28.2 | meq | 60 | meq |
| Ac− | 40 | meq | 37.6 | meq | 80 | meq |

The following considerations are made in order to determine the infusion instructions for the manifest in this example. A total volume of 2130 ml is to be infused over 24 hours:

$$\begin{matrix}Infusion Rate = \frac{Infusion Volume}{Infusion Period} \\ = \frac{2130 ml}{24 hrs} \\ = \frac{88.8 ml}{hr}\end{matrix}$$

Accordingly, each intravenous bag is infused over 12 hours consecutively (88.8 ml/hr), one after the other (intravenous bag 1 of 2 and intravenous bag 2 of 2/24 hour period).

### Third Example

Step 302.

In exemplary step 302, a first selection is received. The first selection comprises the identifier of a premix solution 50 in a plurality of premix solutions (the premix solution list 46). In this example, the premix solution is solution 50-38 of FIG. 5 (CLINIMIX 1800 mL, 90 g AA, 270 g dextrose), and corresponds with a bag type 64 having a volume 68 of 1 L.

Step 304.

In exemplary step 304, a second selection is received. The second selection comprises a first amount of a first addition 58 in the plurality of additions (formulary 56) and a second amount of a second addition 58 in the plurality of additions. In particular, the second selection includes a request for 50 g of lipid, 10 ml of MVI, 1 ml of trace elements, 20 meq of KCl, 40 meq of NaCl, 80 meq of NaAc, and 4.2 meq CaGluc.

Step 306.

In exemplary step 306 and responsive to the first selection, a number of bags 64 of the premix solution needed to satisfy the first selection are reserved from the intravenous bag inventory 62. Based on the first and second selections, two one liter (1 L) bags of CLINIMIX 5/15 are used as the starting formulation. Determinations for an amount of the additions are made as would be required to fulfill the prescription, as set forth below.

Step 308.

It is desired to infuse only 1800 ml (plus additive volume) of the total 2000 ml (plus additive volume) present in two (2) one liter (1 L) bags of plain CLINIMIX. As such only 90% of each CLINIMIX intravenous bag will be infused into the patient. In order to accommodate the discarding of the remaining ten percent of the bag contents, which will also include discarding a portion of the additives, additions must be increased by factor of 1.11. Thus, the following calculations are made.

$$Partial Infusion Factor = \frac{Volume in Container ( without additivites )}{Prescribed Volume ( without additives )} = \frac{2000 ml}{1800 ml} = 1.11$$

Proportioned Up Volume=Partial Infusion Factor*Prescribed Volume

For example, a prescribed 10 ml dose of MVI would be multiplied by the Partial Infusion Factor (10 ml*1.11) resulting in a Proportioned Up Volume of 11.1 ml of MVI.

Proportioned Up Volume=10 ml MVI*1.11=11.1 ml MVI

Note that the proportioned up volume takes into account the prescribed volume and the volume to be discarded. Here, the proportioned up volume consists of 10 ml of MVI intended to be delivered to the patient with an additional 1.1 ml MVI intended to be discarded.

In exemplary step 308, a plurality of additive volumes are determined based on the additions 58 specified in the second selection and the formulary concentrations of these additions. For instance, the plurality of additive volumes comprises a first volume, where the first volume is determined by the quotient of (i) the first amount of the first addition (50 g of lipid) and (ii) the formulary 56 concentration for the first addition in the plurality of additions in the formulary that corresponds to the first addition (20 g/100 ml), multiplied by the partial infusion factor. The plurality of additive volumes comprises a second volume, where the second volume is determined by the quotient of (i) the second amount of the second addition (10 ml of MVI) and (ii) the formulary concentration for the second addition in the plurality of additions in the formulary that corresponds to the second addition (unit dose/10 mL) multiplied by the partial infusion factor. In the present example, step 308 comprises making the calculations set forth in Table 5 below based on the amounts of additions specified in the second request and the corresponding concentrations of such additions in the exemplary formulary 56 illustrated in FIG. 6, in addition to the partial infusion factor:

TABLE 5

| | Total | | |
|---|---|---|---|
| | Volume to | | |
| | be added | | |
| | to | | |
| | CLINIMIX | Volume to | Amount |
| Desired | Bags Using | be added | per |
| Dose | Partial | to each | bag (Dose |

| | Desired | | Formulary | | Converted | Infusion | CLINIMIX | Divided |
|---|---|---|---|---|---|---|---|---|
| Addition | Dose | | Concentration | | to Volume | Factor | Bag | Equally) |
| Lipid | 50 | g | 20 | g/100 ml | 250 ml | 277.5 ml | 138.75 ml | 27.75 g |
| MVI | 10 | ml | Unit Dose | | 10 ml | 11.1 ml | 5.55 ml | 5.55 ml |
| Trace | 1 | ml | Unit Dose | | 1 ml | 1.11 ml | 0.555 ml | 0.555 ml |
| Elements | | | | | | | | |
| KCl | 20 | meq | 2 | meq/ml | 10 ml | 11.1 ml | 5.55 ml | 11.1 meq |
| KCl | | | | | | | | |
| NaCl | 40 | meq | 4 | meq/ml | 10 ml | 11.1 ml | 5.55 ml | 22.2 meq |
| NaCl | | | | | | | | |
| NaAc | 80 | meq | 2 | meq/ml | 40 ml | 44.4 ml | 22.2 ml | 44.4 meq |
| | | | | | | | | Sodium |
| | | | | | | | | Acetate |
| CaGluc | 4.2 | meq | 0.465 | meq/ml | 9 ml | 9.99 ml | 4.995 ml | 2.32 meq |
| | | | | | | | | Calcium |
| | | | | | | | | Gluconate |

| Total | N/A | N/A | 330 ml | 366.3 ml | 183.15 ml | 183.15 ml |
|---|---|---|---|---|---|---|
| Additions | | | | | | |

Step 312.

In exemplary step 312, a manifest 74 for the corresponding intravenous prescription 72 is outputted. This manifest can include, for example, overlabeling, macronutrient ingredients, micronutrient ingredients and infusion directions. Exemplary step 312 is reached when volumes for a premix solution 48 and two or more additions 58 specified by a prescription 72 have been calculated.

Moreover, what is known in step 312 is the number of intravenous bags 70 of the premix solution 48 that will be needed to fill the prescription 72. Specifically, in determining overlabeling for the manifest in this example, it has already been determined that the prescription order will use two intravenous bags of CLINIMIX 5/15 with the additions proportioned up and divided between the two bags. One bag will be infused immediately following the other bag. The total order volume is 2*1000 ml (2 CLINIMIX BAGS)+366.3 ml (additions 58)=2366.3 ml. In order to provide the patient the prescribed TPN, the following partial infusion will need to occur (i) infuse the first 1065 ml from the first 1183.15 ml bag, (ii) discard the remaining 118.15 ml from the first bag, (iii) infuse the second 1065 ml from the second 1183.15 ml bag immediately following the first bag, and (iv) discard the remaining 118.15 ml from the second bag. The patient will have received a total volume of 2130 ml (1800 ml CLINIMIX+330 ml of additive volume) as prescribed.

In determining macronutrient ingredients for the manifest in this example, the final concentration of amino acids is 50 g AA/1183.15 ml=4.23%. The final concentration of dextrose is 150 g dextrose/1183.15 ml=12.68%. The final concentration of lipids is 27.75 g lipids/1183.15 ml=2.35%.

In determining the micronutrient ingredients for the manifest in this example, the amount per bag values are determined based on Table 5 above. The per liter values are calculated according to the formula:

$$\begin{matrix}Per Liter Value = \frac{Amount Per Bag Value}{1.183 L} \\ = 0.845 * Amount Per Bag Value\end{matrix}$$

Using this formula and Table 5, the values for Table 6 below are determined.

TABLE 6

| | | | Amount of | | | | Dose Infused | |
|---|---|---|---|---|---|---|---|---|
| Micro- | | | Dose Infused | | | | Per Day | |
| nutrient | Amount Per Bag | | Per Bag | | Per Liter | | (per 2 bags) | |
| MVI | 5.55 | ml | 5 | ml | 4.69 | ml | 10 | ml |
| Trace | 0.555 | ml | 0.5 | ml | 0.47 | ml | 1 | ml |
| K+ | 11.1 | meq | 10 | meq | 9.38 | meq | 20 | meq |
| Na+ | 66.6 | meq | 60 | meq | 56.28 | meq | 120 | meq |
| Ca++ | 2.33 | meq | 2.1 | meq | 1.97 | meq | 4.2 | meq |
| Cl− | 33.3 | meq | 30 | meq | 28.14 | meq | 60 | meq |
| Ac− | 44.4 | meq | 40 | meq | 37.52 | meq | 80 | meq |

The following considerations are made in order to determine the infusion instructions for the manifest in this example. The total volume available in both bags is 2366.3 ml. A total of 2130 ml is intended to be infused over 24 hours.

$$\begin{matrix}Infusion Rate = \frac{Infusion Volume}{Infusion Period} \\ = \frac{2130 ml}{24 hrs} \\ = \frac{88.75 ml}{hr}\end{matrix}$$

Accordingly, the infusion instructions of this example are to infuse intravenous bag 1 at 88.75 ml/hr for the first twelve hour period (1065 ml infused), then discard the remainder of intravenous bag 1 after the twelve hours infusion. Then infuse intravenous bag 2 at 88.75 ml/hr for the second twelve hour period (1065 ml infused). Then discard remainder of the second intravenous bag after the twelve hours infusion. (Infuse a portion of bag 1 of 2 followed by a portion of bag 2 of 2 consecutively).

It may also be appreciated based on the foregoing example that when determining additive volumes for a plurality of additions, the additive volumes for each respective one of the plurality of additions may affect the calculation of the additive volume of other additions. In this regard, an iterative process may be performed to iteratively determine the additive volume of one or more of the additions.

For example, continuing the third example and using the lipids addition as an example, the desired dose of lipids to be injected is 50 g. As determined in the first iteration described above, the additive volume of the lipids using the partial infusion factor and formulary concentration was 277.5 mL. Multiplying the additive volume by the formulary concentration reveals that a total of 55.5 g of lipids are to be added based on the volume of 277.5 mL of lipids. However, upon determining the additional additive volumes corresponding to the other additions (e.g., the MVI, trace elements, KCl, NaCl, NaAc, and CaGluc), the total final volume of the premix solution with additives added thereto would be 2366.3 mL (e.g., 2000 mL of premix solution and a total volume of 366.3 mL of additions). Thus, the final concentration of lipids present in the premix solution may be determined by dividing 55.5 g present in the solution by the total volume of 2366.3 mL to arrive at a final concentration of lipids in the solution of 0.023 g/mL of lipids. As discussed above, 2130 mL of the premix solution with additives is to be infused. In this regard, multiplying the volume to be infused by the concentration of the lipids in the solution (e.g., 2130 mL multiplied by 0.023 g/mL) reveals that a total of 48.99 g of lipids would be injected, not the specified 50 g of lipids.

In this regard, a second iteration to determine the additive volume of the lipids based on the affect of the volume of the other additives may be undertaken. For example, the final concentration of lipids required in the 2130 mL to be administered can be calculated as the 50 g of the desired dose divided by the total volume of 2130 mL or a concentration of 0.0235 g/mL. This increased concentration may be multiplied by the total volume including all of the additives determined in the first iteration (e.g., 0.0235 g/mL multiplied by 2366.3 mL) to arrive the value of 55.6 g of lipids to be added to the premix solution to account for the added volume attributed to the other additions. Using the formulary concentration, the total volume of lipids to be added now includes 278 mL of lipids as opposed to the 277.5 mL of lipids previously added. As may be appreciated, this additional volume of lipids may also affect the other calculations with respect to the additive volume for the other additions to be added to the premix solution. In this regard, the increased volume of the lipids (e.g., the increase in the total volume of the solution based on the additional lipids to be added) may be used in further iterative calculations of the other additive volumes for the other additions.

It may be appreciated that upon each successive iteration, the change in the determined additive volumes between each successive iteration for each of the additions may be reduced. In this regard, the iterative calculation process of determining the additive volume for the various admissions based on the change in total volume of the solution due to the other additions may be repeated until a steady-state values reach for each of the additive volumes, or the change in additive volumes for any or all of the additions between iterations is below a predetermined threshold (e.g., less than any of the above listed values for the deviation tolerance to overall volume), or any other appropriate metric for determining when the iterative calculations have arrived at a value with acceptable parameters. Furthermore, while one iteration of a single addition as described above, it may be appreciated that iterations with respect to other additions or further iterations may be undertaken in a similar regard as to the one described above.

### Fourth Example

Step 302.

In exemplary step 302, a first selection is received. The first selection comprises the identifier of a premix solution 50 in a plurality of premix solutions (the premix solution list 46). In this example, the premix solution is solution 50-45 of FIG. 5 (CLINIMIX E 2000 mL, 100 g AA, 300 g dextrose).

Step 304.

In exemplary step 304, a second selection is received. The second selection comprises a first amount of a first addition 58 in the plurality of additions (formulary 56) and a second amount of a second addition 58 in the plurality of additions. In particular, the second selection includes a request for 50 g of lipid, 10 ml of MVI, 1 ml of trace elements, 237 meq Na⁺, 108 meq K⁺, 10 meq Mg²⁺, 9 meq Ca²⁺, 185 meq Ac⁻, 108 meq Cl⁻, and 39 mmol PO₄²⁻.

Step 306.

In exemplary step 306 and responsive to the first selection, a number of intravenous bags 64 of the premix solution needed to satisfy the first selection are reserved from the intravenous bag inventory 62. Based on the first and second selections, two one liter (1 L) bags of CLINIMIX 5/15 E are used as the starting formulation. No volume will be removed from the starting bag. Determinations for the amount of the additions will be made as would be required to fulfill the prescription.

Step 308.

In exemplary step 308, a plurality of additive volumes are determined based on the additions 58 specified in the second selection and the formulary concentrations of these additions. For instance, the plurality of additive volumes comprises a first volume, where the first volume is determined by: (i) the first amount of the first addition (50 g of lipid) and (ii) the formulary 56 concentration for the first addition in the plurality of additions in the formulary that corresponds to the first addition (20 g/100 ml). The plurality of additive volumes comprises a second volume, where the second volume is determined by: (i) the second amount of the second addition (10 ml of MVI) and (ii) the formulary concentration for the second addition in the plurality of additions in the formulary that corresponds to the second addition (unit dose/10 mL). In the present example, step 308 comprises making the calculations set forth in Table 7 below based on the amounts of additions specified in the second request and the corresponding concentrations of such additions in the exemplary formulary 56 illustrated in FIG. 6:

TABLE 7

| | Total | | | | | | | | |
|---|---|---|---|---|---|---|---|---|---|
| | Desired | | | | | | Total | Volume to | Amount per |
| | Dose | Present in | Present in | | | Desired | Volume to | be added | Bag (Total |
| | (CLINIMIX | CLINIMIX | 2000 ml | | | Dose | be added to | to each | Desired |
| | E 5/15 + | E 5/15/ | CLINIMIX | Required | Formulary | Converted | CLINIMIX E | CLINIMIX | Dose divided |
| Addition | Additions) | Liter | E 5/15 | Addition | Concentration | to Volume | 5/15 Bags | E 5/15 Bag | equally) |
| Lipid | 50 g | 0 | 0 | 50 g | 20 g/100 ml | 250 ml | 250 ml | 125 ml | 25 g |
| MVI | 10 ml | 0 | 0 | 10 ml | Unit Dose | 10 ml | 10 ml | 5 ml | 5 ml |
| Trace | 1 ml | 0 | 0 | 1 ml | Unit Dose | 1 ml | 1 ml | 0.5 ml | 0.5 ml |
| Na+ | 307 meq | 35 meq | 70 meq | 237 meq | | | | | 153.5 meq |
| | | | | | | | | | Na+ (35 meq + |
| | | | | | | | | | 92.5 meq + |
| | | | | | | | | | 26 meq) |
| | | | | (185 meq | 2 meq/ml | 92.5 ml | 92.5 ml | 46.25 ml | 92.5 meq |
| | | | | NaAc) | | | | | Na+ |
| | | | | (52 meq | 4 meq Na+/ml | 13 ml | 13 ml | 6.5 ml | 26 meq Na+ |
| | | | | NaPhos) | (3 mmol Phos/ | | | | |
| | | | | | ml) | | | | |
| K+ | 168 meq | 30 meq | 60 meq | (108 meq | 2 meq/ml | 54 ml | 54 ml | 27 ml | 84 meq K+ |
| | | | | KCl) | | | | | (30 meq + |
| | | | | | | | | | 54 meq) |
| Mg++ | 20 meq | 5 meq | 10 meq | 10 meq | 4 meq/ml | 2.5 ml | 2.5 ml | 1.25 ml | 10 meq (5 |
| | | | | (Mg | | | | | meq + 5 |
| | | | | Sulfate) | | | | | meq) |
| Ca++ | 18 meq | 4.5 meq | 9 meq | 9 meq | 0.465 | 19.35 ml | 19.35 ml | 9.68 ml | 9 meq (4.5 |
| | | | | | meq/ml | | | | meq + 4.5 |
| | | | | | | | | | meq) |
| Ac− | 345 meq | 80 meq | 160 meq | 185 meq | from NaAc | From NaAc | From NaAc | From NaAc | 172.5 (80 |
| | | | | | above | above | above | above | meq + 92.5 |
| | | | | | | | | | meq) |
| Cl− | 186 meq | 39 meq | 78 meq | 108 meq | from KCl | From KCl | From KCl | From KCl | 93 meq (39 |
| | | | | | above | above | above | above | meq + 54 |
| | | | | | | | | | meq) |
| Total | | | | | | 442.35 ml | 442.35 ml | 221.18 ml | |
| Adds | | | | | | | | | |

Step 312.

In exemplary step 312, a manifest 74 for the corresponding intravenous prescription 72 is outputted. This manifest can include, for example, overlabeling, macronutrient ingredients, micronutrient ingredients and infusion directions. Exemplary step 312 is reached when volumes for a premix solution 48 and two or more additions 58 specified by a prescription 72 have been calculated.

Moreover, what is known in step 312 is the number of intravenous bags 70 of the premix solution 48 that will be needed to fill the prescription 72. Specifically, in determining overlabeling for the manifest in this example, it has already been determined that the prescription order will use two intravenous bags of CLINIMIX E 5/15 with the additions being divided between the two bags. One bag will be infused immediately following the other bag. The total order volume is 2*1000 ml (2 CLINIMIX BAGS)+442.35 ml (additions 58)=2442.35 ml. Each bag will contain 1000 ml (CLINIMIX E)+221.18 ml (½ additions 58)=1221.18 ml total volume.

In determining macronutrient ingredients for the manifest in this example, the final concentration of amino acids is 50 g AA/1221.18 ml=4.09%. The final concentration of dextrose is 150 g dextrose/1221.18 ml=12.28%. The final concentration of lipids is 25.0 g lipids/1221.18 ml=2.05%.

In determining the micronutrient ingredients for the manifest in this example, the amount per bag values are determined based on Table 7 above. The per liter values are calculated according to the formula:

$$\begin{matrix}Per Liter Value = \frac{Amount Per Bag Value}{1221.18 L} \\ = 0.82 * Amount Per Bag Value\end{matrix}$$

Using this formula and Table 7, the values for Table 8 below are determined.

TABLE 8

| | | | | | Total Desired | |
|---|---|---|---|---|---|---|
| | Amount Per Bag | | | | Dose Per Day | |
| | (Total Desired | | | | (2 CLINIMIX E | |
| Micro- | Dose Divided | | | | Bags + | |
| nutrient | Equally) | | Per Liter | | Additions) | |
| MVI | 5 | ml | 4.1 | ml | 10 | ml |
| Trace | 0.5 | ml | 0.41 | ml | 1 | ml |
| Na+ | 153.5 | meq | 125.87 | meq | 307 | meq |
| K+ | 84 | meq | 68.88 | meq | 168 | meq |
| Mg++ | 10 | meq | 8.2 | meq | 20 | meq |
| Ca++ | 9 | meq | 7.38 | meq | 18 | meq |
| Ac− | 172.5 | meq | 141.45 | meq | 345 | meq |
| Cl− | 93 | meq | 76.26 | meq | 186 | meq |
| PO4−− | 34.5 | mmol | 28.29 | mmol | 69 | mmol |

The following considerations are made in order to determine the infusion instructions for the manifest in this example. The total volume of 2442.35 ml is intended to be infused over 24 hours.

$$\begin{matrix}Infusion Rate = \frac{Infusion Volume}{Infusion Period} \\ = \frac{2442.35 ml}{24 hrs} \\ = \frac{101.76 ml}{hr}\end{matrix}$$

Accordingly, the infusion instructions of this example are to infuse each bag over twelve hours consecutively (101.76 ml/hr), one after the other (bag 1 of 2 and bag 2 of 2/24 hour period).

### Fifth Example

Step 302.

In exemplary step 302, a first selection is received. The first selection comprises the identifier of a premix solution 50 in a plurality of premix solutions (the premix solution list 46). In this example, the premix solution is solution 50-38 of FIG. 5 (CLINIMIX 1800 mL, 90 g AA, 270 g dextrose), and corresponds with a bag type 64 having a volume 68 of 2 L.

Step 304.

In exemplary step 304, a second selection is received. The second selection comprises a first amount of a first addition 58 in the plurality of additions (formulary 56) and a second amount of a second addition 58 in the plurality of additions. In particular, the second selection includes a request for 50 g of lipid, 10 ml of MVI, 1 ml of trace elements, 20 meq of KCl, 40 meq of NaCl, 80 meq of NaAc, and 4.2 meq CaGluc.

Step 306.

In exemplary step 306 and responsive to the first selection, a number of bags 64 of the premix solution needed to satisfy the first selection may be optionally reserved from the intravenous bag inventory 62. In this example, based on the first selection and optionally the second selection, one two liter (2 L) bag of CLINIMIX 5/15 is used as the starting formulation.

In this regard, selection of a single two liter (2 L) bag of CLINIMIX 5/15, as opposed to two one liter (1 L) bags of CLINIMIX 5/15 may reduce the compounding steps entailed for filling a prescription as well as subsequent administration steps.

Step 308.

It is desired to infuse a minimum volume of only 1800 ml (plus additive volume) of the total 2000 ml (plus additive volume) present in the one (1) two liter (2 L) bag of plain CLINIMIX. As such only 90% of the single CLINIMIX intravenous bag will be infused into the patient. In order to accommodate the discarding of the remaining ten percent of the bag contents, which will also include discarding a portion of the additives, additions must be increased by factor of 1.11. Thus, the following calculations are made.

$$Partial Infusion Factor = \frac{Volume in Container ( without additivites )}{Prescribed Volume ( without additives )} = \frac{2000 ml}{1800 ml} = 1.11$$

Proportioned Up Volume=Partial Infusion Factor*Prescribed Volume

For example, a prescribed 10 ml dose of MVI would be multiplied by the Partial Infusion Factor (10 ml*1.11) resulting in a Proportioned Up Volume of 11.1 ml of MVI.

Proportioned Up Volume=10 ml MVI*1.11=11.1 ml MVI

Note that the proportioned up volume takes into account the prescribed volume and the volume to be discarded. Here, the proportioned up volume consists of 10 ml of MVI intended to be delivered to the patient with an additional 1.1 ml MVI intended to be discarded.

In exemplary step 308, a plurality of additive volumes are determined based on the additions 58 specified in the second selection and the formulary concentrations of these additions. For instance, the plurality of additive volumes comprises a first volume, where the first volume is determined by the quotient of (i) the first amount of the first addition (50 g of lipid) and (ii) the formulary 56 concentration for the first addition in the plurality of additions in the formulary that corresponds to the first addition (20 g/100 ml), multiplied by the partial infusion factor. The plurality of additive volumes comprises a second volume, where the second volume is determined by the quotient of (i) the second amount of the second addition (10 ml of MVI) and (ii) the formulary concentration for the second addition in the plurality of additions in the formulary that corresponds to the second addition (unit dose/10 mL) multiplied by the partial infusion factor. In the present example, step 308 comprises making the calculations set forth in Table 9 below based on the amounts of additions specified in the second request and the corresponding concentrations of such additions in the exemplary formulary 56 illustrated in FIG. 6, in addition to the partial infusion factor:

TABLE 9

| | | | | | | | Total Volume | | | |
|---|---|---|---|---|---|---|---|---|---|---|
| | | | | | Desired | | to be added to | | | |
| | | | | | Dose | | CLINIMIX Bag | | | |
| | | | Formulary | | Converted | | Using Partial | | | |
| Addition | Desired Dose | | Concentration | | to Volume | | Infusion Factor | | Amount in bag | |
| Lipid | 50 | g | 20 | g/100 ml | 250 | ml | 277.5 | ml | 55.5 | g |
| MVI | 10 | ml | Unit Dose | | 10 | ml | 11.1 | ml | 11.1 | ml |
| Trace Elements | 1 | ml | Unit Dose | | 1 | ml | 1.11 | ml | 1.11 | ml |
| KCl | 20 | meq | 2 | meq/ml | 10 | ml | 11.1 | ml | 22.2 | meq KCl |
| NaCl | 40 | meq | 4 | meq/ml | 10 | ml | 11.1 | ml | 88.8 | meq NaCl |
| NaAc | 80 | meq | 2 | meq/ml | 40 | ml | 44.4 | ml | 88.8 | meq Sodium |
| | | | | | | | | | | Acetate |
| CaGluc | 4.2 | meq | 0.465 | meq/ml | 9 | ml | 9.99 | ml | 4.64 | meq Calcium |
| | | | | | | | | | | Gluconate |

| | | | | | | | | |
|---|---|---|---|---|---|---|---|---|
| Total | N/A | N/A | 330 | ml | 366.3 | ml | 366.3 | ml |
| Additions | | | | | | | | |

Step 312.

In exemplary step 312, a manifest 74 for the corresponding intravenous prescription 72 is outputted. This manifest can include, for example, overlabeling, macronutrient ingredients, micronutrient ingredients and infusion directions. Exemplary step 312 is reached when volumes for a premix solution 48 and two or more additions 58 specified by a prescription 72 have been calculated.

Moreover, what is known in step 312 is the number of intravenous bags 70 of the premix solution 48 that will be needed to fill the prescription 72. Specifically, in determining overlabeling for the manifest in this example, it has already been determined that the prescription order will use one intravenous bag of CLINIMIX 5/15 with the additions identified above. The total order volume is 2000 ml (1 CLINIMIX 2 L BAG)+366.3 ml (additions 58)=2366.3 ml. In order to provide the patient the prescribed TPN, the following partial infusion will need to occur (i) infuse the 2130 ml from the bag, (ii) discard the remaining 236.3 ml.

In determining macronutrient ingredients for the manifest in this example, the final concentration of amino acids is 100 g AA/2366.3 ml=4.23%. The final concentration of dextrose is 300 g dextrose/2366.3 ml=12.68%. The final concentration of lipids is 55.5 g lipids/2366.3 ml=2.35%.

In determining the micronutrient ingredients for the manifest in this example, the amounts are determined based on Table 9 above. The per liter values are calculated according to the formula:

$$\begin{matrix}Per Liter Value = \frac{Amount In Bag Value}{2.368 L} \\ = 0.845 * Amount In Bag Value\end{matrix}$$

Using this formula and Table 9, the values for Table 10 below are determined.

TABLE 10

| | | | Amount of | | | | | |
|---|---|---|---|---|---|---|---|---|
| Micro- | | | Dose Infused | | | | Dose Infused | |
| nutrient | Amount In Bag | | From Bag | | Per Liter | | Per Day | |
| MVI | 11.1 | ml | 10 | ml | 4.69 | ml | 10 | ml |
| Trace | 11.1 | ml | 1 | ml | 0.47 | ml | 1 | ml |
| K+ | 22.2 | meq | 20 | meq | 9.38 | meq | 20 | meq |
| Na+ | 133.2 | meq | 120 | meq | 56.28 | meq | 120 | meq |
| Ca++ | 4.66 | meq | 4.2 | meq | 1.97 | meq | 4.2 | meq |
| Cl− | 66.6 | meq | 60 | meq | 28.14 | meq | 60 | meq |
| Ac− | 88.8 | meq | 80 | meq | 37.52 | meq | 80 | meq |

The following considerations are made in order to determine the infusion instructions for the manifest in this example. The total volume available in the bag is 2366.3 ml. A total of 2130 ml is intended to be infused over 24 hours.

$$\begin{matrix}Infusion Rate = \frac{Infusion Volume}{Infusion Period} \\ = \frac{2130 ml}{24 hrs} \\ = \frac{88.75 ml}{hr}\end{matrix}$$

Accordingly, the infusion instructions of this example are to infuse the intravenous bag 1 at 88.75 ml/hr for the twenty four hour period (2130 ml infused), then discard the remainder of intravenous bag 1.

### Sixth Example

In this example, computer system 10 may comprise or otherwise operatively interface with one or more programs, or applications, that facilitate the generation and entry of an order for a parenteral or enteral preparation (e.g., an order or prescription), via interactive user interface screens accessible at user interface 32. Additionally, computer system 10 may include a formulary 56 comprising both a plurality of additions 58 and one or a plurality of premix solutions 48, wherein each premix solution 48 has a corresponding identifier 50 and a corresponding bag volume 52 associated therewith. In some implementations, a premix solution identifier 50 may be selected directly or by usage of a pre-established order template stipulation inclusion of a given premix solution identifier 50, wherein upon user entry of a volume (e.g., a desired volume of premix solution to be administered to a patient), a premix solution 48 having a corresponding bag volume 52 sufficient to provide the entered volume may be automatically selected for inclusion in the preparation order.

By way of example, reference is again made to FIG. 1A and FIG. 1B, which each illustrate eight rows of different premix solutions 48, with each row including two premix solutions 48 having different bag volumes (i.e., 1000 ml and 2000 ml) and being of a common type (i.e., having common compound component concentrations). In this example, one or more of the sixteen types of premix solutions identified in FIG. 1A and FIG. 1B may be included in formulary 56, wherein each premix solution 48 has a corresponding bag volume 52 (i.e., 1 L or 2 L). In conjunction with this example, formulary 56 may include at least two premix solutions 48 of a common type (i.e., having common compound concentrations) and having different corresponding bag volumes 52 (e.g., 1 L and 2 L). A common premix solution identifier 50 may be utilized in connection with premix solutions of a common type.

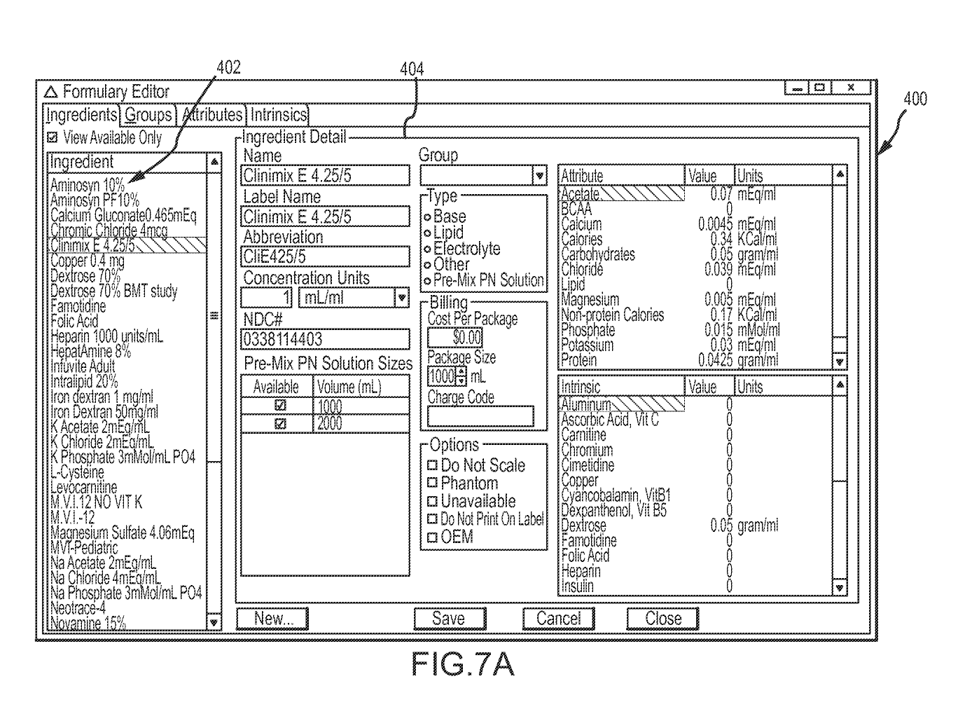

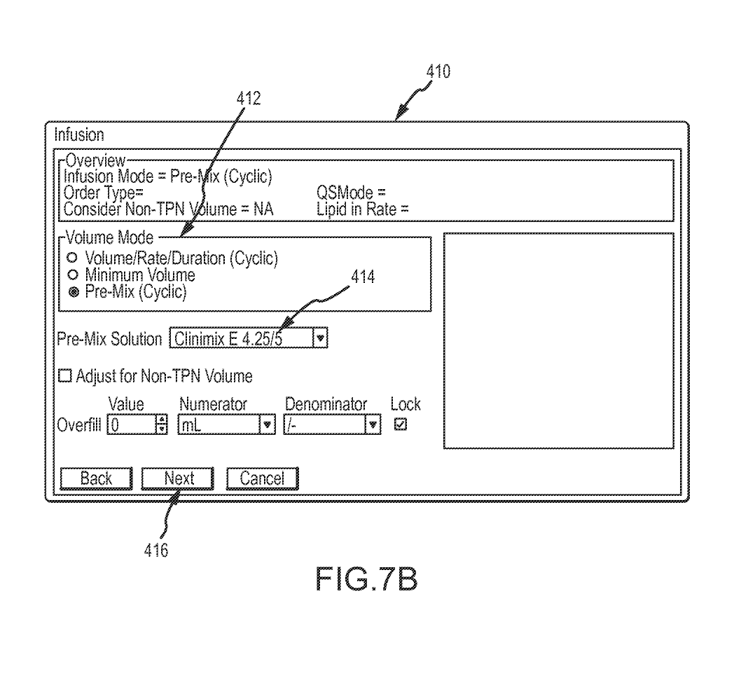

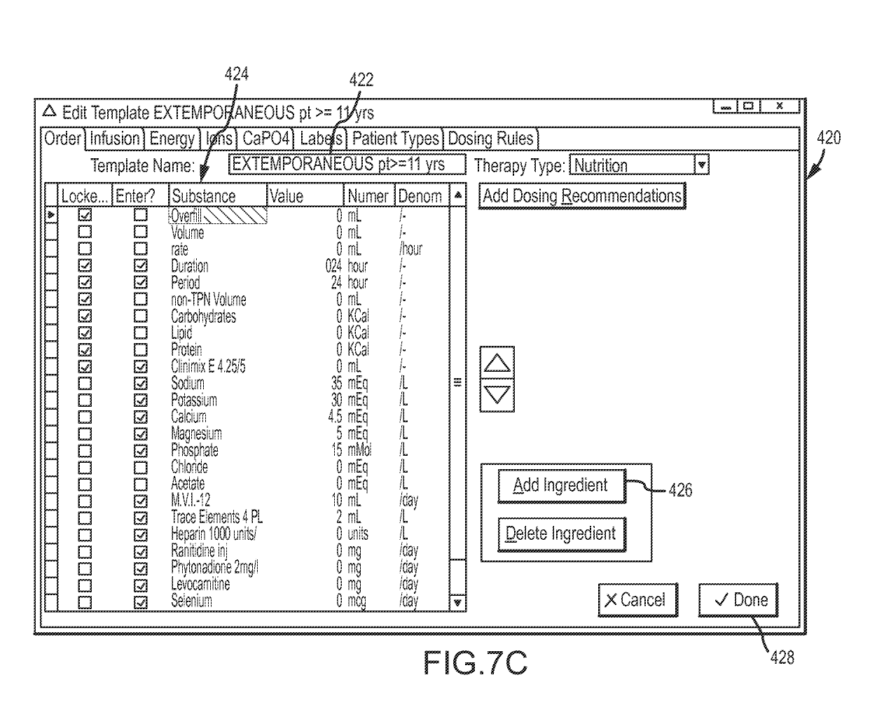

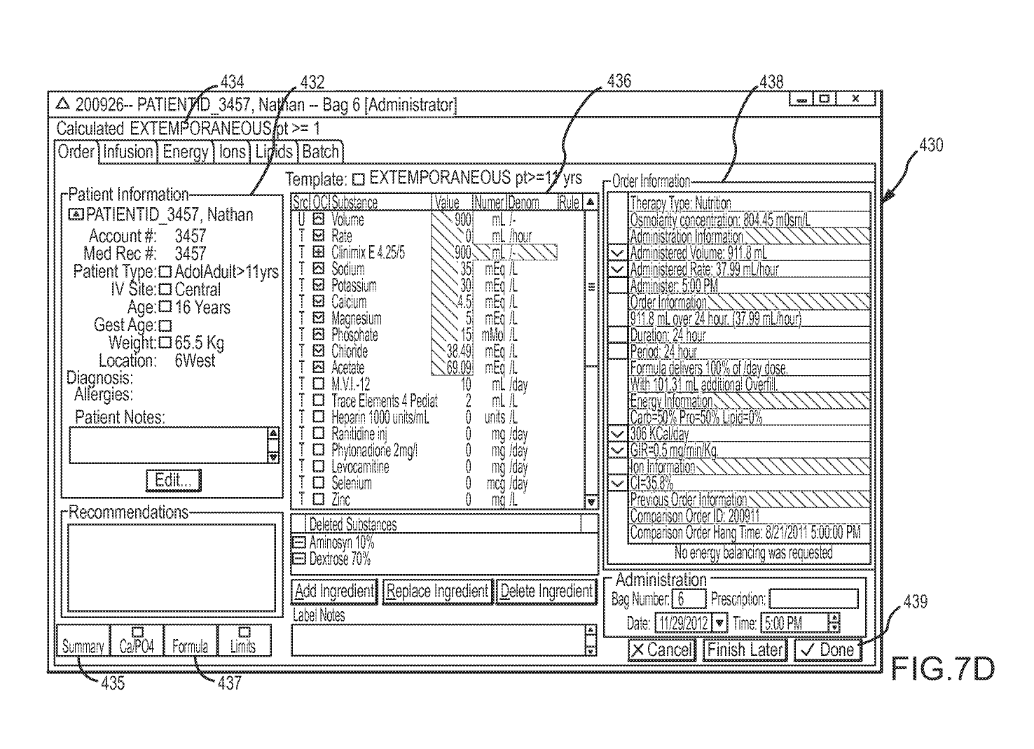

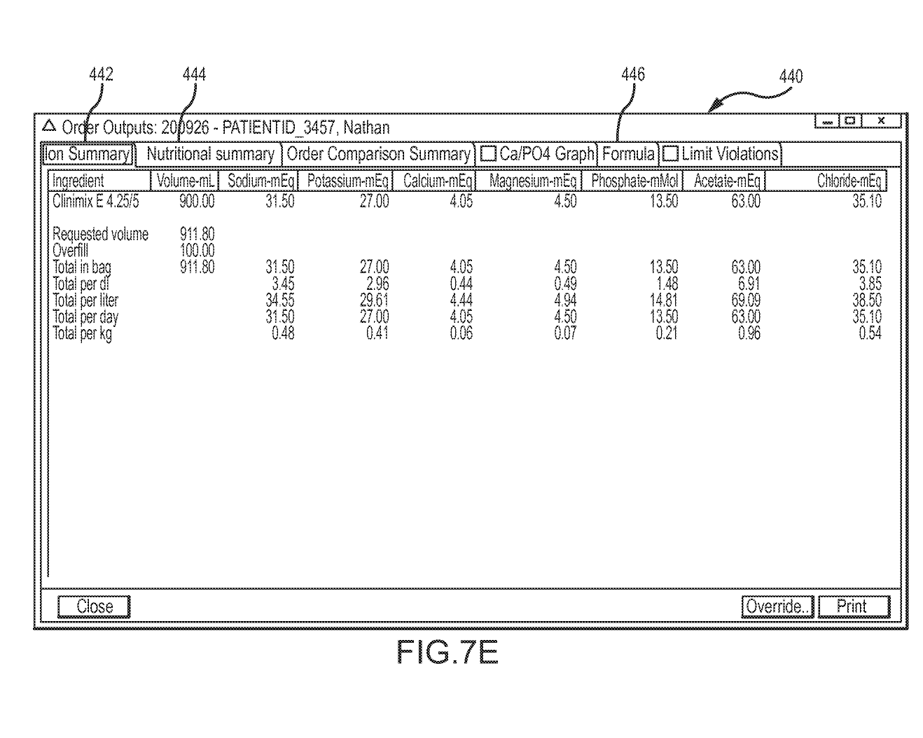

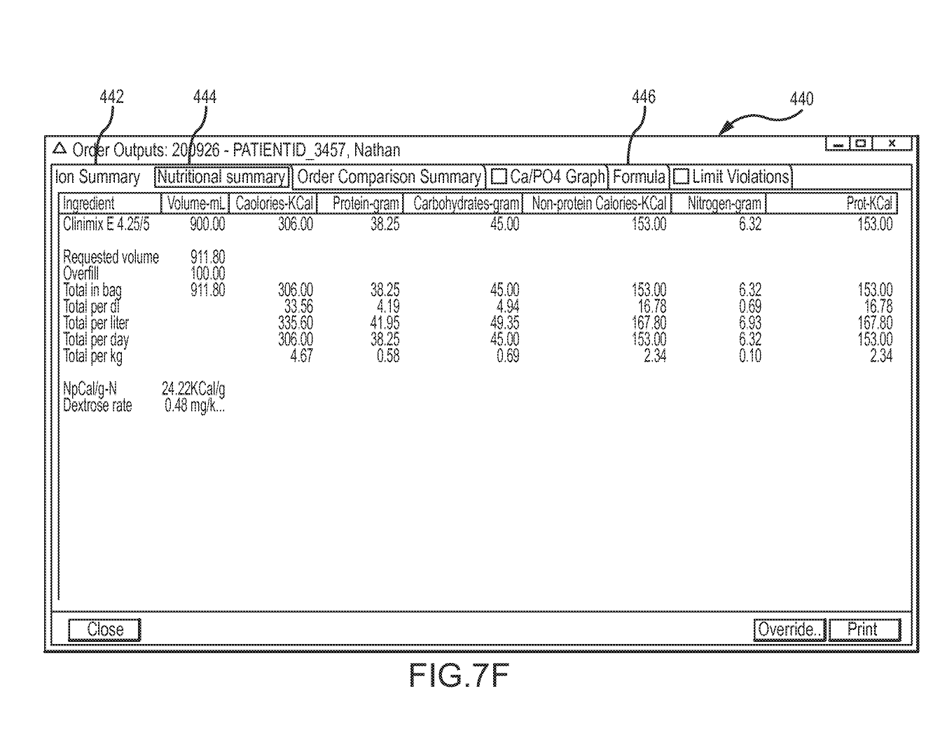

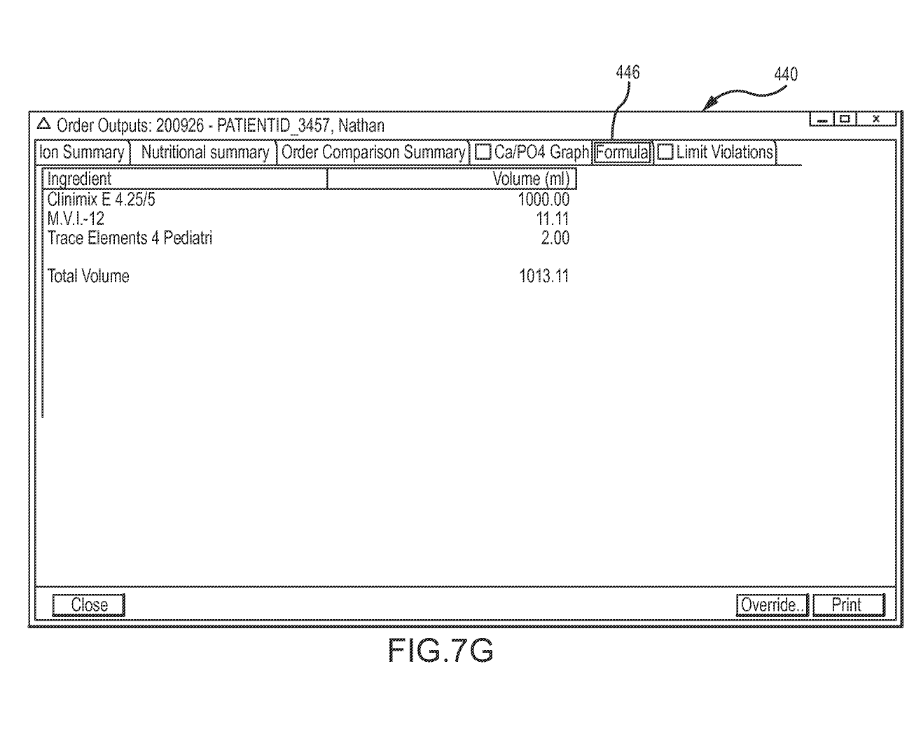

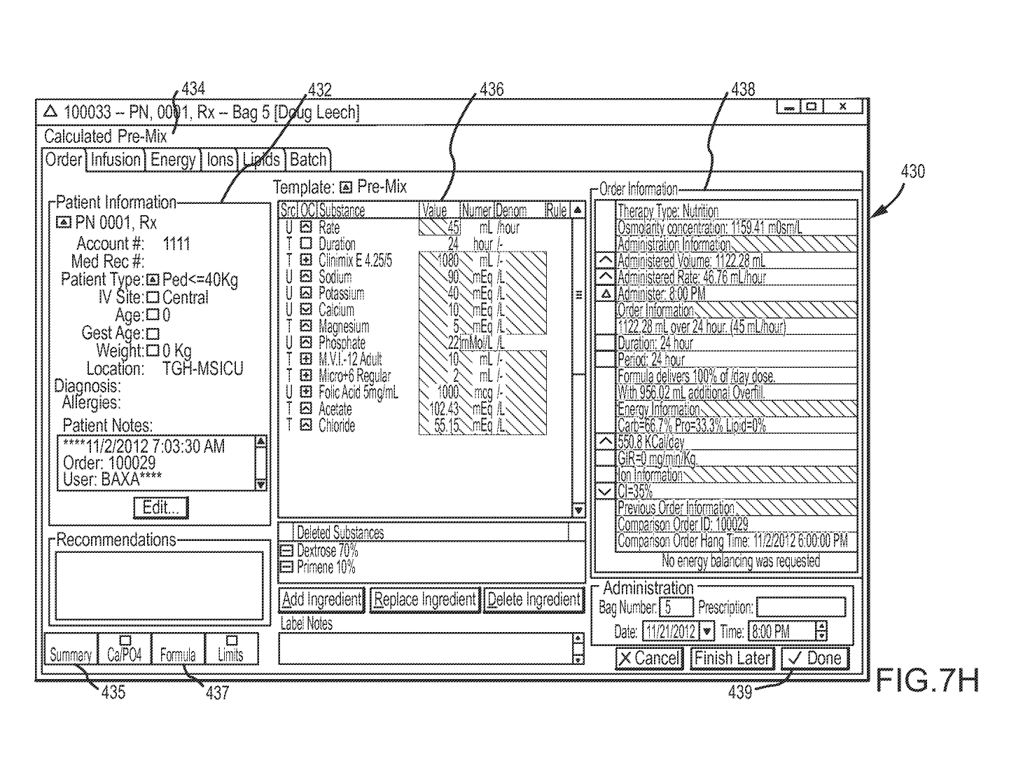

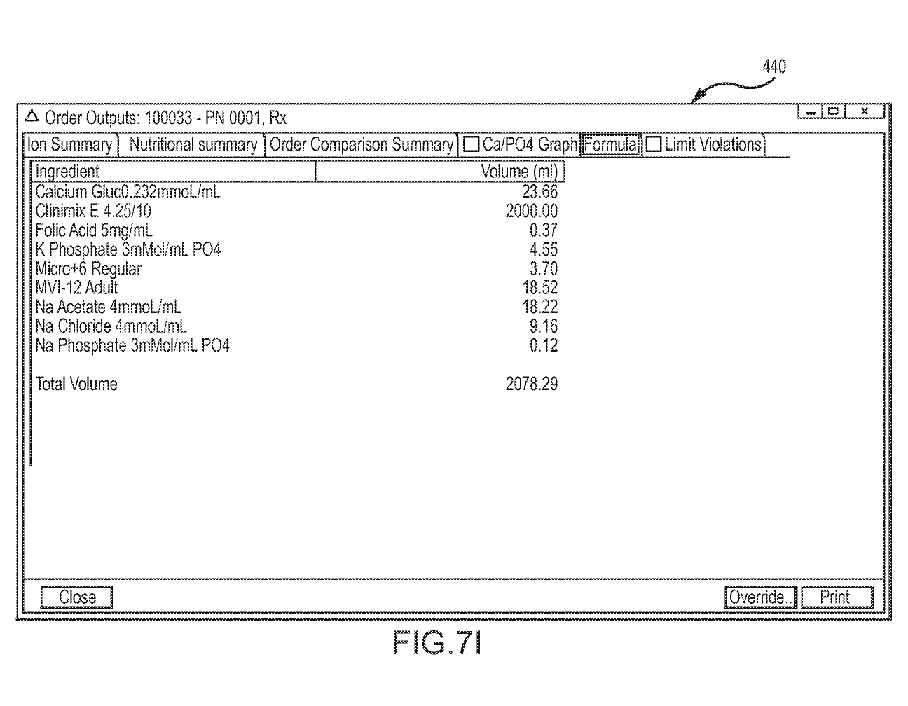

Reference is now made to FIGS. 7A-7I which illustrate various prescription order entry screens that may be provided at and operatively interacted with by a user at user interface 32 of computer system 10. As shown by interactive use interface screen 400 of FIG. 7A, a formulary 56 may have a corresponding ingredient listing in scrollable ingredient listing box 402 that includes a premix solution identifier 50 for a given type of premix solution 48 (e.g., CLINIMIX E4 4.25/5 in the illustrated example). Upon selection of such premix solution identifier 50 in listing box 402, an “Ingredient Detail” portion 404 may indicate that corresponding premix solutions 48 are includable, or available, in two corresponding bag volumes (e.g., CLINIMIX E 4.25/5 in 1 L bag and CLINIMIX E 4.25/5 in 2 L bag in the illustrated example).

In some implementations, the preparation order generation and entry program(s) may facilitate the generation and entry of a preparation order that includes a premix solution 48 via the establishment and use of an order template that specifies the use of a given type of premix solution 48. For example, and as illustrated by FIG. 7B, interactive user interface screen 410 may be accessed by a user and an order type (e.g., entitled “Pre-Mix (Cyclic)”) may be selected from an order type listing 412. In conjunction with such selection, a user may select a given type of premix solution 48 comprising formulary 56 utilizing scrollable selection box 414. For example, if a user desires to establish an order template for a preparation order utilizing a premix solution type having a premix solution identifier 50 (e.g., “CLINIMIX E 4.25/5” in the illustrated example), the user may utilize box 414 to select such premix solution type then utilize the “Next” button 416 to access one or more additional interactive user interface screens to establish additional infusion parameters (e.g., volume, rate, parameters; 2-in-1 or 3-in-1 parameters), and to access interactive user interface screen 420, shown in FIG. 7C to complete the order template.

The interactive user interface screen 420 may be utilized to name a given order template via user input box 422 and to identify and/or modify amounts of certain items for inclusion in a given order template via scrollable listing box 424. Note that listing box 424 includes the given premix solution identifier 50 corresponding with the given order template (e.g., “CLINIMIX E 4.25/5” in the illustrated example). As shown, selected ones of the items in listing box 424 may be locked to preclude modification of such items during subsequent use of the order template, including for example the corresponding premix solution identifier 50. As further illustrated, amounts may be set forth for certain items in the listing corresponding with compounds contained within the corresponding given premix solution 48 (e.g., amounts of sodium potassium, calcium, magnesium and phosphate in the illustrated example). As indicated, in addition to such included items, additional ingredients, or additions, may be included in the order template (e.g., by accessing a formulary listing via interactive use of button 426 (e.g., the additions of “M.V.I.-12” and “Trace Elements” in the illustrated example). Further, amounts of such additions may be established in the order template via interactive use of listing box 424 (e.g., for clicking upon an adjacent cell and entering a value). When a user has completed, or established, a given order template, such order template may be saved for subsequent use in conjunction with a given specific preparation order via use of interactive button 428.

In this regard, the prescription order entry program(s) may be provided so that one or more user interface screens may be accessed by a user to select a given one of one or a plurality of pre-established order templates, wherein a user may select an order template that includes a premix solution identifier 50 that corresponds with a type of premix solution 48 includable for use in one or more corresponding bag volumes (e.g., 1 L and/or 2 L). By way of example, and in relation to the exemplary formulary shown in the interactive user interface screen 400 of FIG. 7A and exemplary order template shown in the user interface screen 420 of FIG. 7C, reference is now made to FIG. 7D.

FIG. 7D illustrates an interactive user interface screen 430 displaying an order template accessed for use in generation and entry of a given preparation order (e.g., for a given patient indicated in the “Patient Information” screen portion 432). Note that, the name of the order template being utilized may be displayed in display box 434. As shown, an interactive listing box 436 may be employed to establish a desired, or prescribed, amount of the corresponding premix solution 48 (i.e., corresponding with the premix solution identifier 50 established in the order template) to be administered in use of the corresponding fulfilled order. For example, a user may enter a “Volume” value, or “Rate” and “Duration” values via use of adjacent interactive cells, to establish the desired to-be-administered volume of the premix solution (e.g., 900 mL of CLINIMIX E 4.25/5 in the illustrated example).

The interactive listing box 436 may also be employed to modify amounts of ingredients, or additions, for inclusion in the order. In the illustrated example, the amounts of additions for “M.V.I.-12” (i.e., 10 mL/day) and “Trace Elements” (i.e., 2 mL/L) have not been modified in the order shown in FIG. 7D relative to the amounts of such additions included in the order template shown in FIG. 7C. The preparation order generation and entry program(s) may be provided so that the amounts of one or more of such additions may be automatically computed and increased in order formulation calculations to account for dilution. Such calculations may utilize the Partial Infusion Factor and Proportion Volume Calculation Techniques described hereinabove. In the illustrated example, a calculated amount of “M.V.I.-12” may be 11.11 mL (i.e., 1000 mL/900 mL×10 mL); and, since “Trace Elements” is specified with a volume-based denominator, a corresponding calculated volume of “Trace Elements” may be based on the desired volume (i.e., 900 mL), such that the calculated amount may be 2.00 mL (i.e., (900 mL/1000 mL×2 mL)×(1000 mL/900 mL)); to yield a total volume of 1013.11 mL.

The preparation order generation and entry program(s) may be provided so that upon entry of the desired volume of premix solution, one of a plurality of corresponding available bag types, or volumes, may be automatically selected for inclusion in the order. For example, in relation to the formulary example of FIG. 7A, for an order template employing “CLINIMIX E 4.25/5”, the preparation order generation and entry program(s) may automatically select a corresponding premix solution bag having a volume of 1 L or a corresponding premix solution bag having a volume of 2 L. Such selection may entail a comparison of the desired volume of premix with one or more of the volumes corresponding with the volumes of the different bags available for inclusion in the order. In one approach, the desired volume may be compared with the volume of the smallest corresponding available bag (e.g., 1 L), wherein if the desired volume is smaller the smallest bag is selected, and if the desired volume is greater, a larger volume bag (e.g., 2 L) is selected (e.g., a bag having a volume that is greater than the desired volume).

Further, the preparation order generation and entry program(s) may be provided so as to automatically calculate an overfill volume in relation to a given order. Such overfill volume may be automatically calculated to include an amount equal to the difference between the desired volume of premix solution (e.g., 900 mL in the illustrated example) and the volume of the bag of the premix solution selected for inclusion in the order (e.g., 1000 mL in the illustrated example). Such overfill volume may be further automatically calculated to account for the volume of any additions (i.e., added ingredients) included in the order.

In this regard, the preparation order generation and entry program(s) may be provided so that interactive user interface screen 430 includes an “Order Information” portion 438 that provides order information to a user regarding the automatically calculated overfill volume, and regarding an automatically calculated volume of fluid that may be administered upon order fulfillment and use. For the order example shown in FIG. 7D, the user interface screen 430 indicates a calculated “Administered Volume” of 911.8 mL and a calculated “Overfill” volume of 101.31 mL. Such order information may be included in a manifest output corresponding with the order after entry, e.g., for use in compounding and/or inclusion in bag label generation/printing. In that regard, when a user is satisfied with the contents of a given order reflected by interactive user screen 430, the user may proceed to enter, or complete, the order by use of the interactive button 439.

In addition to the foregoing, a user may utilize interactive button 435 to access interactive user interface screen 440 shown in FIGS. 7E, 7F, and 7G. As shown in FIGS. 7E, 7F, and 7G, selectable tabs 442, 444, and 446 may be utilized to obtain various order content summaries, including a summary of the preparation order formula.

Reference is now made to FIG. 7H which illustrates interactive user screen 430 in relation to another exemplary order. In this example, a user has selected the use of a template that stipulates inclusion of a premix solution 48 identified as “CLINIMIX E 4.25/10”. The user has further input a desired volume of the premix solution 48 by entering a “Rate” of 45 mL/hour and “Duration” of 24 hours. In turn, the preparation order generation and entry program(s) has automatically calculated a volume of the premix solution to be 1080 mL. In the illustrated example, the compound components of the premix are identified by unit amounts per liter (e.g., sodium, potassium, calcium, magnesium, and phosphate). In addition, the illustrated order reflects the inclusion of the following additions:
“MVI-12 Adult”, “Micro+6 Regular”, “Folic Acid 5 mg/mL”, “Acetate”, and “Chloride”.

In conjunction with the generation of the order illustrated in FIG. 7H, the preparation order generation and entry program(s) may provide for the automatic selection of a two liter bag of the stipulated premix solution 48 corresponding with the order template utilized for order entry. In turn, to account for the difference between the ordered amount of premix solution (i.e., 1080 mL) and the volume of the bag (i.e., 2 L), included amounts of certain ones of the additions may automatically be increased by computational formula calculations to account for dilution effects. In particular, the automated formula calculations may increase the amounts of the following additions having specified unit amounts that are not based on amounts per unit volume:
“MVI-12 Adult”, “Micro+6 Regular”, and “Folic Acid 5 mg/mL”.
   Such calculations may utilize the Partial Infusion Factor and Proportion Volume Calculation Techniques described hereinabove.

As shown in the “Order Information” 438 portion of the interactive user screen 430 of FIG. 7E, the automatically calculated “Administered Volume” is 1122.28 mL, and the automatically calculated “Overfill Volume” to be included in the bag as formulated is 956.02 mL.

In conjunction with the exemplary order of FIG. 7H, the final calculated formulation amounts to be included in the order may be accessed by a user via use of the interactive button 437 entitled “Formula”. Upon such interaction, a user may access the interactive user interface screen 440 shown in FIG. 7I. As shown in FIG. 7I, the final amounts of the given premix solution, compounds included therein, and additions thereto are listed. As may be appreciated, the information displayed in user interface screens 430 and 440 described above may be included on a manifest, or order output, for storage, inclusion on one or more labels, and/or for use in conjunction with compounding steps.

### CONCLUSION

All references cited herein are incorporated herein by reference in their entirety and for all purposes to the same extent as if each individual publication or patent or patent application was specifically and individually indicated to be incorporated by reference in its entirety herein for all purposes.

Plural instances may be provided for components, operations or structures described herein as a single instance. Boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first contact could be termed a second contact, and, similarly, a second contact could be termed a first contact, which changing the meaning of the description, so long as all occurrences of the “first contact” are renamed consistently and all occurrences of the second contact are renamed consistently. The first contact and the second contact are both contacts, but they are not the same contact.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method for determining an amount of an ingredient for preparation of a requested therapy to be administered to a patient, comprising:
receiving a first selection of a premix solution from a plurality of premix solutions and an ordered amount of the premix solution, wherein each respective one of the premix solutions includes a predetermined description at least comprising:
(i) an identifier for the respective premix solution,
(ii) a volume of the respective premix solution in a prepackaged bag containing the premix solution, and
(iii) an amount of a first component in the respective premix solution contained in the prepackaged bag;
receiving a second selection, wherein the second selection includes a first amount of a first addition from a plurality of additions, wherein the plurality of additions are predefined in a formulary comprising at least one formulary concentration for each respective addition in the plurality of additions; and
determining an additive volume for the first addition, to be added to the prepackaged bag containing the premix solution, at least partially based on the ordered amount, the volume of the premix solution in the prepackaged bag, the first amount, and the formulary concentration for the first addition, wherein the ordered amount is less than the volume of the premix solution in the prepackaged bag, and the determining further comprises:
calculating a partial infusion factor based on the ordered amount and the volume of the premix solution in the prepackaged bag, wherein the partial infusion factor is determined by dividing the volume of the premix solution in the prepackaged bag by the ordered amount;
wherein the additive volume is determined at least partially based on the partial infusion factor and comprises a proportioned up volume determined by multiplying the first amount of the first addition by the partial infusion factor.

2.-3. (canceled)

4. The method of claim 1, wherein the ordered amount is larger than any one of the volumes of the plurality of premix solutions in the prepackaged bags, and wherein the method further comprises:
calculating at least one aliquot volume for the additive volume among a plurality of prepackaged bags of the premix solution and;
reserving, in response to the first selection, a number of prepackaged bags of the premix solution needed to satisfy the first selection from a bag inventory;
wherein the at least one aliquot volume is determined by dividing the additive volume by the number of prepackaged bags.

5.-7. (canceled)

8. The method of claim 4, further comprising:
establishing a draw down amount for each one of the number of prepackaged bags reserved.

9. The method of claim 4, further comprising:
calculating an administration rate for a corresponding bag within the number of prepackaged bags that have been reserved.

10. The method of claim 1, wherein the plurality of premix solutions comprise at least a first premix solution and a second premix solution that have a common corresponding identifier and a different corresponding volume, wherein the first selection comprises the common identifier corresponding with the first premix solution and the second premix solution in the plurality of premix solutions, and wherein the method further comprises:
selecting automatically, responsive to the first selection, at least one of the first premix solution or at least one of the second premix solution for use in the determining step.

11. The method of claim 4, wherein the second selection further includes a second amount of a second addition from the plurality of additions, and the determining includes:
determining an additive volume for the second addition, to be added to the prepackaged bag containing the premix solution, at least partially based on the additive volume for the first addition, the ordered amount, the volume of the premix solution in the prepackaged bag, the second amount, and the formulary concentration for the second amount;
wherein the additive volume for the first addition and the additive volume for the second addition affect the ordered amount, and wherein the determining step further includes iteratively solving for the additive volume of the first and second additions based on the affect of the additive volume for the first addition and the additive volume for the second addition on the ordered amount.

12. (canceled)

13. The method of claim 1, wherein the first selection and the second selection are in the requested therapy and wherein the requested therapy is electronically received through a data input device and is entered into a computer system.

14.-15. (canceled)

16. The method of claim 13, wherein the data input device comprises an interactive user interface, and wherein the method further comprises:
receiving user input via use of at least one order template at an interactive interface to enter the requested therapy.

17. The method of claim 1, further comprising:
outputting a manifest output corresponding to the preparation comprising at least one of:
contents of a corresponding bag, including a final amount of the first compound and a final amount of the first addition in the corresponding bag;
instructions for adding at least one aliquot volume for the additive volume among a plurality of prepackaged bags of the premix solution; or
instructions for withdrawing a draw down amount from at least one prepackaged bag of premix solution.

18. (canceled)

19. The method of claim 17, wherein the method further comprises:
preparing an overlabel for a prepackaged bag of premix solution comprising the manifest output;
wherein each respective overlabel specifies an expiration date for the preparation and an administration rate for a corresponding bag.

20.-23. (canceled)

24. The method of claim 1, wherein an addition in the plurality of additions is a medication, a trace element, a vitamin, an electrolyte, an IV lipid emulsion, an IV lipid microemulsion, a specialty amino acid, or a nutriceutical ingredient, and wherein the premix solution comprises an amount of a second component in the respective premix solution, and wherein the first component is an amino acid and the second component is dextrose or carbohydrate.

25. (canceled)

26. A computer based system for determining an amount of an ingredient for preparation for a requested therapy to be administered to a patient, comprising:
a data input device executable by a processor of the computer based system that is operable to:
receive a first selection of a premix solution from a plurality of premix solutions and an ordered amount of the premix solution, wherein each respective one of the premix solutions includes a predetermined description at least comprising:
(i) an identifier for the respective premix solution,
(ii) a volume of the respective premix solution in a prepackaged bag containing the premix solution, and
(iii) an amount of a first component in the respective premix solution contained in the prepackaged bag, and
receive a second selection, wherein the second selection includes a first amount of a first addition from a plurality of additions, wherein the plurality of additions are predefined in a formulary comprising at least one formulary concentration for each respective addition in the plurality of additions; and
a preparation processing module executed by a processor of the computer based system that is operable to determine an additive volume for the first addition, to be added to the prepackaged bag containing the premix solution, at least partially based on the ordered amount, the volume of the premix solution in the prepackaged bag, the first amount, and the formulary concentration for the first addition.

27. The system of claim 26, wherein the ordered amount is less than the volume of the premix solution in the prepackaged bag, and wherein the preparation processing module is operable to calculate a partial infusion factor by dividing the volume of the premix solution in the prepackaged bag by based on the ordered amount, and wherein the additive volume is at least partially determined by multiplying the first amount of the first addition by the partial infusion factor.

28.-34. (canceled)

35. The system of claim 26, wherein the plurality of premix solutions comprise at least a first premix solution and a second premix solution that have a common corresponding identifier and a different corresponding volume, wherein the first selection comprises the common identifier corresponding with the first premix solution and the second premix solution in the plurality of premix solutions, and wherein the preparation processing module is operable to select automatically, responsive to the first selection, at least one of the first premix solution or at least one of the second premix solution for use by the preparation processing module to determine the additive volume of the first addition.

36. The system of claim 27, wherein the second selection further includes a second amount of a second addition from the plurality of additions, and the preparation processing module is operable to determine an additive volume for the second addition, to be added to the prepackaged bag containing the premix solution, at least partially based on the additive volume for the first addition, the ordered amount, the volume of the premix solution, in the prepackaged bag, the second amount, and the formulary concentration for the second amount, and wherein the additive volume for the first addition and the additive volume for the second addition affect the ordered amount, and wherein the preparation processing module is operable to iteratively solve for the additive volume of the first and second additions based on the affect of the additive volume for the first addition and the additive volume for the second addition on the ordered amount.

37. (canceled)

38. The system of claim 26, wherein the first selection and the second selection are in the requested therapy and wherein the requested therapy is electronically received by the computer based system through an interactive user interface comprising the data input device, wherein the interactive user interface is operable to receive user input via use of at least one order template at an interactive interface to enter the requested therapy.

39.-40. (canceled)

41. The system of claim 26, further comprising:
a manifest output corresponding to the preparation comprising at least one of:
contents of a corresponding bag, including a final amount of the first compound and a final amount of the first addition in the corresponding bag;
instructions for adding at least one aliquot volume for the additive volume among a plurality of prepackaged bags of the premix solution; or
instructions for withdrawing a draw down amount from at least one prepackaged bag of premix solution.

42. (canceled)

43. The system of claim 41, further comprising:
an overlabel for a prepackaged bag of premix solution comprising the manifest output, wherein each respective overlabel specifies an expiration date for the preparation and an administration rate for a corresponding bag.

44.-47. (canceled)

48. The system of claim 26, wherein an addition in the plurality of additions is a medication, a trace element, a vitamin, an electrolyte, an IV lipid emulsion, an IV lipid microemulsion, a specialty amino acid, or a nutriceutical ingredient, and wherein the premix solution comprises an amount of a second component in the respective premix solution, and wherein the first component is an amino acid and the second component is dextrose or carbohydrate.

49.-154. (canceled)
